SEQUENCE LISTING (48 sequences)

SEQ ID NO: 1 - protein, 115 aa, Mus musculus
EVKLEESGGGLVQPGGSMKLSCAASGFTFSDAWMDWVRQSPEKGLEWVAEIGNKGNNHATYYAESVKGRFTVSRDDSKSRVYLQMNSLRVEDTGTYYCTTRFAYWGQGTLVTVSA

SEQ ID NO: 2 - protein, 115 aa, Artificial Sequence
  synthesized
EVKLEESGGGLVQPGGSMKLSCAASGFTFSDAWMDWVRQSPEKGLEWVAEIGNKGNNHATNYAESVKGRFTVSRDDSKSRVYLQMNSLRVEDTGTYYCTTRFAYWGQGTLVTVSA

SEQ ID NO: 3 - protein, 115 aa, Artificial Sequence
  synthesized
KVKLEESGGGLVQPGGSMKLSCAASGFTFSDAWMDWVRQSPEKGLEWVAEIGNKGNNHATNYAESVKGRFTVSRDDSKSRVYLQMNSLRVEDTGTYYCTTRFAYWGQGTPVTVPA

SEQ ID NO: 4 - protein, 107 aa, Mus musculus
DIKMTQSPSSMYASLGERVTITCKASQDINSYLSWFQQKPGKSPKTLIYRANRLVDGVPSRFSGSGSGQDYSLTISSLEYEDMGIYYCLQYDEFPRTFGGGTKLEIK

SEQ ID NO: 5 - protein, 107 aa, Artificial Sequence
  synthesized
DIKMTQSPSSMYASLGERVTIPCKASQDINSYLSWFQQKPGKSPKTLIYRANRLVDGVPSRFSGSGSGQDYSLTISSLEYEDMGIYYCLQYDEFPRTFGGGTKLEIK

SEQ ID NO: 6 - protein, 10 aa, Artificial Sequence
  synthesized
GFTFSDAWMD

SEQ ID NO: 7 - protein, 16 aa, Artificial Sequence
  synthesized
NKGNNHATYYAESVKG

SEQ ID NO: 8 - protein, 4 aa, Artificial Sequence
  synthesized
RFAY

SEQ ID NO: 9 - protein, 11 aa, Artificial Sequence
  synthesized
KASQDINSYLS

SEQ ID NO: 10 - protein, 7 aa, Artificial Sequence
  synthesized
RANRLVD

SEQ ID NO: 11 - protein, 10 aa, Artificial Sequence
  synthesized
LQYDEFPRTF

SEQ ID NO: 12 - protein, 16 aa, Artificial Sequence
  synthesized
NKGNNHATNYAESVKG

SEQ ID NO: 13 - DNA, 345 bp, Artificial Sequence
  synthesized
aaagtcaagctagaagaaagcggtggcggacttgttcagcctgggggatccatgaagttatcttgcgccgcatccggttttaccttttctgatgcctggatggattgggtgagacaatctccagaaaaaggtctggaatgggtagccgaaattggtaacaagggcaataatcacgccacgaactatgccgagtctgtaaaaggaagattcactgtttccagagacgattcaaagtccagagtctatctacaaatgaattcattaagagtcgaagatactggtacttactactgtaccactaggtttgcatattggggtcaaggtacaccggtgacggttcctgcc

SEQ ID NO: 14 - DNA, 321 bp, Artificial Sequence
  synthesized
gacattaaaatgactcagagtccatcctcaatgtacgcttccttaggagaaagagtcactatcccttgcaaggcatcccaagatatcaactcatacttgagttggttccaacaaaaaccaggtaaatctcctaaaaccctgatttatcgtgccaataggcttgtggatggcgttccatccagattcagcggatcaggtagtgggcaagattattctttgacaatttcctcattggaatacgaagatatgggcatatactattgcttacagtatgatgagtttccacgtacttttggcggtggaacaaaattagaaatcaaa

SEQ ID NO: 15 - protein, 237 aa, Artificial Sequence
  Synthesized
KVKLEESGGGLVQPGGSMKLSCAASGFTFSDAWMDWVRQSPEKGLEWVAEIGNKGNNHATNYAESVKGRFTVSRDDSKSRVYLQMNSLRVEDTGTYYCTTRFAYWGQGTPVTVPAGGGGSGGGGSGGGGSDIKMTQSPSSMYASLGERVTIPCKASQDINSYLSWFQQKPGKSPKTLIYRANRLVDGVPSRFSGSGSGQDYSLTISSLEYEDMGIYYCLQYDEFPRTFGGGTKLEIK

SEQ ID NO: 16 - protein, 5 aa, Artificial Sequence
  Synthesized
GGGGS

SEQ ID NO: 17 - protein, 110 aa, Artificial Sequence
  Synthesized
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 18 - protein, 38 aa, Artificial Sequence
  Synthesized
QVRKAAITSYEKSDGVYTGLSTRNQETYETLKHEKPPQ

SEQ ID NO: 19 - protein, 22 aa, Artificial Sequence
  Synthesized
MDFQVQIFSFLLISASVIMSRG

SEQ ID NO: 20 - protein, 417 aa, Artificial Sequence
  Synthesized
MDFQVQIFSFLLISASVIMSRGKVKLEESGGGLVQPGGSMKLSCAASGFTFSDAWMDWVRQSPEKGLEWVAEIGNKGNNHATNYAESVKGRFTVSRDDSKSRVYLQMNSLRVEDTGTYYCTTRFAYWGQGTPVTVPAGGGGSGGGGSGGGGSDIKMTQSPSSMYASLGERVTIPCKASQDINSYLSWFQQKPGKSPKTLIYRANRLVDGVPSRFSGSGSGQDYSLTISSLEYEDMGIYYCLQYDEFPRTFGGGTKLEIKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 21 - DNA, 711 bp, Artificial Sequence
  Synthesized
aaagtcaagctagaagaaagcggtggcggacttgttcagcctgggggatccatgaagttatcttgcgccgcatccggttttaccttttctgatgcctggatggattgggtgagacaatctccagaaaaaggtctggaatgggtagccgaaattggtaacaagggcaataatcacgccacgaactatgccgagtctgtaaaaggaagattcactgtttccagagacgattcaaagtccagagtctatctacaaatgaattcattaagagtcgaagatactggtacttactactgtaccactaggtttgcatattggggtcaaggtacaccggtgacggttcctgccggaggtggcggtagcggaggcggcggttctggaggtggcgggagcgacattaaaatgactcagagtccatcctcaatgtacgcttccttaggagaaagagtcactatcccttgcaaggcatcccaagatatcaactcatacttgagttggttccaacaaaaaccaggtaaatctcctaaaaccctgatttatcgtgccaataggcttgtggatggcgttccatccagattcagcggatcaggtagtgggcaagattattctttgacaatttcctcattggaatacgaagatatgggcatatactattgcttacagtatgatgagtttccacgtacttttggcggtggaacaaaattagaaatcaaa

SEQ ID NO: 22 - DNA, 330 bp, Artificial Sequence
  Synthesized
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatct

SEQ ID NO: 23 - DNA, 117 bp, Artificial Sequence
  Synthesized
caagtgcgaaaggcagctataaccagctatgagaaatcagatggtgtttacacgggcctgagcaccaggaaccaggagacttacgagactctgaagcatgagaaaccaccacagtag

SEQ ID NO: 24 - DNA, 66 bp, Artificial Sequence
  Synthesized
atggattttcaggtgcagattttcagcttcctgctaatcagtgcctcagtcataatgtccagagga

SEQ ID NO: 25 - DNA, 1254 bp, Artificial Sequence
  Synthesized
atggattttcaggtgcagattttcagcttcctgctaatcagtgcctcagtcataatgtccagaggaaaagtcaagctagaagaaagcggtggcggacttgttcagcctgggggatccatgaagttatcttgcgccgcatccggttttaccttttctgatgcctggatggattgggtgagacaatctccagaaaaaggtctggaatgggtagccgaaattggtaacaagggcaataatcacgccacgaactatgccgagtctgtaaaaggaagattcactgtttccagagacgattcaaagtccagagtctatctacaaatgaattcattaagagtcgaagatactggtacttactactgtaccactaggtttgcatattggggtcaaggtacaccggtgacggttcctgccggaggtggcggtagcggaggcggcggttctggaggtggcgggagcgacattaaaatgactcagagtccatcctcaatgtacgcttccttaggagaaagagtcactatcccttgcaaggcatcccaagatatcaactcatacttgagttggttccaacaaaaaccaggtaaatctcctaaaaccctgatttatcgtgccaataggcttgtggatggcgttccatccagattcagcggatcaggtagtgggcaagattattctttgacaatttcctcattggaatacgaagatatgggcatatactattgcttacagtatgatgagtttccacgtacttttggcggtggaacaaaattagaaatcaaaggtggaggtggatccggtggaggtggatccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag

SEQ ID NO: 26 - protein, 8 aa, Artificial Sequence
  Synthesized
WSHPQFEK

SEQ ID NO: 27 - DNA, 24 bp, Artificial Sequence
  Synthesized
tggagccacccgcagttcgagaaa

SEQ ID NO: 28 - protein, 425 aa, Artificial Sequence
  Synthesized
MDFQVQIFSFLLISASVIMSRGKVKLEESGGGLVQPGGSMKLSCAASGFTFSDAWMDWVRQSPEKGLEWVAEIGNKGNNHATNYAESVKGRFTVSRDDSKSRVYLQMNSLRVEDTGTYYCTTRFAYWGQGTPVTVPAGGGGSGGGGSGGGGSDIKMTQSPSSMYASLGERVTIPCKASQDINSYLSWFQQKPGKSPKTLIYRANRLVDGVPSRFSGSGSGQDYSLTISSLEYEDMGIYYCLQYDEFPRTFGGGTKLEIKWSHPQFEKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 29 - DNA, 1278 bp, Artificial Sequence
  Synthesized
atggattttcaggtgcagattttcagcttcctgctaatcagtgcctcagtcataatgtccagaggaaaagtcaagctagaagaaagcggtggcggacttgttcagcctgggggatccatgaagttatcttgcgccgcatccggttttaccttttctgatgcctggatggattgggtgagacaatctccagaaaaaggtctggaatgggtagccgaaattggtaacaagggcaataatcacgccacgaactatgccgagtctgtaaaaggaagattcactgtttccagagacgattcaaagtccagagtctatctacaaatgaattcattaagagtcgaagatactggtacttactactgtaccactaggtttgcatattggggtcaaggtacaccggtgacggttcctgccggaggtggcggtagcggaggcggcggttctggaggtggcgggagcgacattaaaatgactcagagtccatcctcaatgtacgcttccttaggagaaagagtcactatcccttgcaaggcatcccaagatatcaactcatacttgagttggttccaacaaaaaccaggtaaatctcctaaaaccctgatttatcgtgccaataggcttgtggatggcgttccatccagattcagcggatcaggtagtgggcaagattattctttgacaatttcctcattggaatacgaagatatgggcatatactattgcttacagtatgatgagtttccacgtacttttggcggtggaacaaaattagaaatcaaatggagccacccgcagttcgagaaaggtggaggtggatccggtggaggtggatccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacagtag

SEQ ID NO: 30 - protein, 5 aa, Artificial Sequence
  synthesized
DAWMD

SEQ ID NO: 31 - protein, 19 aa, Artificial Sequence
  synthesized
EIGNKGNNHATYYAESVKG

SEQ ID NO: 32 - protein, 10 aa, Artificial Sequence
  synthesized
GGGGSGGGGS

SEQ ID NO: 33 - protein, 15 aa, Artificial Sequence
  synthesized
GGGGSGGGGSGGGGS

SEQ ID NO: 34 - protein, 20 aa, Artificial Sequence
  synthesized
GGGGSGGGGSGGGGSGGGGS

SEQ ID NO: 35 - DNA, 45 bp, Artificial Sequence
  Synthesized
ggaggtggcggtagcggaggcggcggttctggaggtggcgggagc

SEQ ID NO: 36 - protein, 19 aa, Artificial Sequence
  synthesized
EIGNKGNNHATNYAESVKG

SEQ ID NO: 37 - protein, 237 aa, Artificial Sequence
  Synthesized
EVKLEESGGGLVQPGGSMKLSCAASGFTFSDAWMDWVRQSPEKGLEWVAEIGNKGNNHATYYAESVKGRFTVSRDDSKSRVYLQMNSLRVEDTGTYYCTTRFAYWGQGTLVTVSAGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPRTFGGGTKLEIK

SEQ ID NO: 38 - protein, 417 aa, Artificial Sequence
  Synthesized
MDFQVQIFSFLLISASVIMSRGEVKLEESGGGLVQPGGSMKLSCAASGFTFSDAWMDWVRQSPEKGLEWVAEIGNKGNNHATYYAESVKGRFTVSRDDSKSRVYLQMNSLRVEDTGTYYCTTRFAYWGQGTLVTVSAGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPRTFGGGTKLEIKGGGGSGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQ

SEQ ID NO: 39 - protein, 25 aa, Artificial Sequence
  synthesized
KVKLEESGGGLVQPGGSMKLSCAAS

SEQ ID NO: 40 - protein, 30 aa, Artificial Sequence
  synthesized
KVKLEESGGGLVQPGGSMKLSCAASGFTFS

SEQ ID NO: 41 - protein, 17 aa, Artificial Sequence
  synthesized
WVRQSPEKGLEWVAEIG

SEQ ID NO: 42 - protein, 14 aa, Artificial Sequence
  synthesized
WVRQSPEKGLEWVA

SEQ ID NO: 43 - protein, 11 aa, Artificial Sequence
  synthesized
WGQGTPVTVPA

SEQ ID NO: 44 - protein, 23 aa, Artificial Sequence
  Synthesized
DIKMTQSPSSMYASLGERVTIPC

SEQ ID NO: 45 - protein, 32 aa, Artificial Sequence
  Synthesized
RFTVSRDDSKSRVYLQMNSLRVEDTGTYYCTT

SEQ ID NO: 46 - protein, 15 aa, Artificial Sequence
  Synthesized
WFQQKPGKSPKTLIY

SEQ ID NO: 47 - protein, 32 aa, Artificial Sequence
  Synthesized
GVPSRFSGSGSGQDYSLTISSLEYEDMGIYYC

SEQ ID NO: 48 - protein, 9 aa, Artificial Sequence
  Synthesized
GGGTKLEIK

(19) United States
(12) Patent Application Publication
(10) Pub. No.: US 2023/0058044 A1
(43) Pub. Date: Feb. 23, 2023

(54) CHIMERIC ANTIGEN RECEPTOR TO CARBOHYDRATE ANTIGENS

(71) Applicants: RAMOT AT TEL-AVIV UNIVERSITY LTD., Tel Aviv (IL); ICHILOV TECH LTD., Tel Aviv (IL); YEDA RESEARCH AND DEVELOPMENT CO. LTD., Rehovot (IL)

(72) Inventors: Vered PADLER-KARAVANI, Tel Aviv (IL); Yafit ATIYA-NASAGI, Tel Aviv (IL); Ron AMON, Tel Aviv (IL); Anat GLOBERSON LEVIN, Tel Aviv (IL); Tova WAKS, Petach Tikva (IL); Moran RAWET-SLOBODKIN, Tel Aviv (IL); Lihi NINIO MANY, Tel Aviv (IL); Zelig ESHHAR, Tel Yitzak (IL)

(21) Appl. No.: 17/779,704

(22) PCT Filed: Nov. 25, 2020
(86) PCT No.: PCT/IL2020/051215

§ 371 Date: May 25, 2022

Related U.S. Application Data
(60) Provisional application No. 62/940,274, filed on Nov. 26, 2019.
(60) Provisional application No. 63/069,051, filed on Aug. 23, 2020.

(51) Int. Cl. A61K 35/17; C07K 14/725; C07K 14/705; C07K 14/735; C12N 5/0783

(52) U.S. Cl. CPC A61K 35/17; C07K 14/7051; C07K 14/70521; C07K 14/70535; C12N 5/0636; A61K 2039/5156

(57) ABSTRACT

The present invention discloses chimeric antigen receptors that specifically recognize and bind to SLe A carbohydrate antigen with high specificity and selectivity. The invention further provides lymphocytic cells, such as T cells, comprising said CARs, compositions comprising said cells or CARs as well as uses thereof.

### FIELD OF THE INVENTION

The present invention relates to chimeric antigen receptors (CARs) that specifically recognize and bind to SLeA carbohydrate antigen, cells expressing said CARs, compositions comprising said cells or CARs as well as uses thereof.

### BACKGROUND OF THE INVENTION

Cancer is a leading cause of death worldwide and selective targeting by therapeutic monoclonal antibodies show increasing success in modern oncology. The variable domains of such potent antibodies are also used to engineer T cells to express chimeric antigen receptors (CAR) for immunotherapy. Genetically engineered CAR are modular single chain structures that contain a single-chain of an antibody variable domains (scFv) as an extracellular targeting unit, connected by a flexible hinge to a transmembrane domain followed by an intracellular co-stimulatory and signaling domains. Immunotherapy with CAR T cells is a promising technology currently most successful in targeting CD19 for hematological B cell malignancies. This had prompted the evaluation of other potential targets, including several clinical trials for treatments of solid tumors with CAR targeting EGFR (NCT02666248) and CEA (NCT02416466). Currently, the most commonly used antibodies and CAR are targeting proteins on cancer cells. While cell surface glycosylation is a common feature of all living cells, its expression pattern is commonly altered on cancer cells leading to variations in the basic core carbohydrate chains (glycans), also conjugated to glycoproteins and glycolipids (Varki et al., 2009, Essentials of Glycobiology). Oncogenic glycosylation is limited to a distinct subset of tumor-associated carbohydrate antigens (TACAs) that are selectively and abundantly expressed on cancer cells. Sialic acids are acidic sugars that cover cell surfaces glycans and glycoconjugates. Their expression patterns are frequently altered on cancer cells (Padler-Karavani, V., 2014, Cancer Lett 352, 102-112), and often correlate with advanced stage, progression and/or metastasis (Brooks et al., 2008, Anticancer Agents Med Chem 8, 2-21). Thus, sialylated-TACA are important targets for cancer therapy and diagnostics (Padler-Karavani et al., 2011, Cancer Res 71, 3352-3363). Several glycan-targeting antibodies had been developed over the years, and some had been examined for CAR immunotherapy with limited success. Yet a potential obstacle for using anti-carbohydrate antibodies is their low affinity and low specificity compared to antibodies targeting proteins (Haji-Ghassemi et al., Glycobiology 25, 920-952; Manimala et al., 2007, Glycobiology 17, 17C-23C). Currently, only a single antibody targeting TACA is in clinical use—Dinutuximab, an anti-GD2 antibody for neuroblastoma therapy.

The sialyl-Lewis A (SLeA) tetrasaccharide (Neu5Acα2-3Galβ1-3(Fucα1-4)GlcNAβ1-R) (Ugorski et al., Acta Biochim Pol. 2002; 49: 303-311; Padler-Karavani, 2014), which is detected by the serological assay as CA19-9, is a sialic acid-containing cancer-associated marker widely used to monitor clinical response to therapy (Ballehaninna and Chamberlain, 2012; J Gastrointest Oncol 3, 105-119). Antibody clone named 1116NS19.9, that recognizes SLeA was developed already in 1979 by Koprowski et al. 9 (Koprowski et al., Somatic Cell Genet. 1979; 5: 957-971). Decades later, this antibody is used in many available kits to determine SLeA levels in cancer patients. However, it cannot be used for cancer treatment due to its low affinity.

There is an urgent need for therapeutic agents targeting SLeA glycan which is expressed in many cancer types. Such agents could potentially be used for the treatment of a wide range of cancer types. Yet, despite the facts that it is known for many years that SLeA is expressed on cancer cells, there is no available treatment based on the recognition of this target.

### SUMMARY OF THE INVENTION

The present invention discloses chimeric antigen receptors (CARs) comprising an antigen binding domain that recognize and binds specifically to a Sialyl Lewis A glycan. In particular, disclosed are CARs having the antigen binding domain comprising a heavy-chain variable domain (VH) having amino acid sequence as set forth in SEQ ID NO: 3 and a light-chain variable domain having amino acid sequence as set forth in SEQ ID NO: 5. T-cells comprising said CAR were shown to selectively bind to Sialyl Lewis A glycan but not to its close structural analog SLeX. It was also shown in xenograft model of pharynx squamous cell carcinoma that these T-cells reduce the tumor size upon local or systemic administration.

According to one aspect, the present invention provides a chimeric antigen receptor (CAR) comprising an antigen binding domain that binds specifically to Sialyl Lewis A glycan (SLeA), wherein the antigen binding domain comprises three complementarity determining regions (CDRs) and four framework (FR) domains of a heavy-chain variable domain (VH) having amino acid sequence as set forth in SEQ ID NO: 1 or an analog thereof and three CDRs and four framework (FR) domains of a light-chain variable domain (VL) having amino acid sequence as set forth in SEQ ID NO: 4, or an analog thereof, wherein the analog has at least 90% sequence identity to said sequences. According to one embodiment, the CDR2 of the VH domain (VH-CDR2) comprises amino acid sequence SEQ ID NO: 7. According to one embodiment, the CDR2 of the VH domain comprises amino acid sequence SEQ ID NO: 31. According to some embodiments, the VH-CDR1 comprises amino acid sequence selected from SEQ ID NOs: 6 and 30. According to another embodiment, the VH-CDR2 comprises at least one non-conservative substitution. According to some embodiments, the VH-CDR2 comprises amino acid sequence selected from 7 and 31. According to some embodiments, the CDRs 1, 2, and 3 of the VH domain of the CAR of the present invention comprise amino acid sequences SEQ ID NOs: 6, 12 and 8, respectively, and CDRs 1, 2, and 3 of the VL domain comprise amino acid sequences SEQ ID NOs: 9, 10 and 11, respectively. According to other embodiments, the amino acid sequences of CDRs of the VH domain are set forth in SEQ ID NOs: 30, 36 and 8, and the amino acid sequences of CDRs of the VL domain are set forth in SEQ ID NOs: 9, 10, and 11. According to some embodiments, the VH domain of the CAR of the present invention has amino acid sequence set forth in SEQ ID NO: 2. According to some embodiments, the antigen binding domain further comprises at least 2 non-conservative substitutions of amino acids in framework sequences of the VH domain and/or of VL domain. According to some embodiments, the antigen binding domain comprises 2, 3, or 4 non-conservative substitutions of amino acids in the framework sequences. According to certain embodiments, the non-conservative substitutions is for proline amino acid residue.

According to some embodiments, one or more substitution(s) is in a framework sequence selected from VH-FR1, VH-FR4, VL-FR1 and any combination thereof. According to another embodiment, the VH-CDR 1, 2 and 3 comprise amino acid sequences SEQ ID NOs: 30, 12, and 8, respectively, the VL-CDRs 1, 2 and 3 comprise amino acid sequences SEQ ID NOs: 9, 10 and 11, respectively, VH-FRs 1, 2 and 4 comprises acid sequences SEQ ID NOs: 39, 42 and 43, respectively, and the VL-FR 1 comprises acid sequences SEQ ID NO: 44. According to some embodiments, the VH-CDR 1 comprises amino acid sequence selected from SEQ ID NOs: 30 and 6 and the VH-CDR2 comprises amino acid sequence selected from SEQ ID NO: 12 and 36. According to certain embodiments, the CDRs 1, 2, and 3 of the VH domain comprises amino acid sequences SEQ ID NOs: 30, 36 and 8, respectively, the CDRs 1, 2, and 3 of the VL domain comprise amino acid sequences SEQ ID NOs: 9, 10 and 11, the VH-FRs 1, 2 and 4 comprise amino acid sequences SEQ ID NOs: 40, 42 and 43, respectively, and the VL-FR1 comprises acid sequences SEQ ID NO: 44. According to some embodiments, the CDRs 1, 2, and 3 of the VH domain consist of amino acid sequences SEQ ID NOs: 30, 36 and 8, respectively, the CDRs 1, 2, and 3 of the VL domain consist of amino acid sequences SEQ ID NOs: 9, 10, and 11, the VH-FRs 1, 2 and 4 consist of amino acid sequences SEQ ID NOs: 40, 42 and 43, respectively, and the VL-FR1 consists of acid sequences SEQ ID NO: 44. According to some embodiments the CDRs 1, 2, and 3 of the VH domain comprises amino acid sequences SEQ ID NOs: 30, 36 and 8, respectively, the CDRs 1, 2, and 3 of the VL domain comprise amino acid sequences SEQ ID NOs: 9, 10 and 11, the VH-FRs 1, 2, 3 and 4 comprise amino acid sequences SEQ ID NOs: 40, 42, 45 and 43, respectively, and the VL-FR1, 2, 3 and 4 comprise amino acid sequences SEQ ID NOs: 44, 46, 47 and 48, respectively. According to some embodiments the CDRs 1, 2, and 3 of the VH domain consist of amino acid sequences SEQ ID NOs: 30, 36 and 8, respectively, the CDRs 1, 2, and 3 of the VL domain consist of amino acid sequences SEQ ID NOs: 9, 10 and 11, the VH-FRs 1, 2, 3 and 4 consist of amino acid sequences SEQ ID NOs: 40, 42, 45 and 43, respectively, and the VL-FR1, 2, 3 and 4 consist of amino acid sequences SEQ ID NOs: 44, 46, 47 and 48, respectively.

According to some embodiments, the VH domain of the CAR comprises amino acid sequence SEQ ID NO: 3 and/or the VL domain comprises amino acid sequence SEQ ID NO: 5. According to other embodiments, the VH domain and/or VL domain of the CAR further comprise one or more conservative substitutions in the framework sequence(s), wherein the resulted VH domain has at least 90% sequence identity to SEQ ID NO: 3 and/or the resulted VL domain has at least 90% sequence identity to SEQ ID NO: 5. According to some embodiments, the VH and the VL domains are linked by a spacer to form a single chain variable fragment (scFv). According to some embodiments, the antigen binding domain of the CAR comprises amino acid sequence SEQ ID NO: 15 or 37 or an analog thereof having at least 90/o sequence identity to said sequence.

The CAR of the present invention comprises a transmembrane domain (TM domain), and a costimulatory domain and/or an activation domain. According to some embodiments, the TM domain is a TM domain of a receptor selected from CD28 and CD8, or an analog thereof having at least 85% amino acid identity to the original sequence and/or the costimulatory domain is selected from a costimulatory domain of a protein selected from CD28, 4-1BB, OX40, iCOS, CD27, CD80, and CD70, an analog thereof having at least 85% amino acid identity to the original sequence and any combination thereof, and/or the activation domain is selected from FcRγ and CD3-ζ activation domains. According to some embodiments, the CAR comprises a leading peptide. According to some embodiments, the CAR comprises or consists of amino acid sequence SEQ ID NO: 20. According to another embodiment, the CAR comprises or consists of amino acid sequence SEQ ID NO: 28. According to yet another embodiment, the CAR comprises or consists of amino acid sequence SEQ ID NO: 38.

According to some embodiments, the CAR comprises a scFv comprising the binding site that binds specifically to SLeA, a TM domain and a costimulatory domain of CD28, and an activation domain selected from FcRγ and CD3-ζ activation domains. According to specific embodiments, the CAR comprises a scFv comprising the amino acid sequence set forth in SEQ ID NO: 22, a TM domain selected from a TM domain of a receptor selected from CD28 and CD8, a costimulatory domain selected from CD28, 4-1BB, OX40, iCOS, CD27, CD80, CD70, an analog thereof and any combination thereof, and an activation domain selected from FcRγ and CD3-ζ activation domain.

According to another aspect, the present invention provides a nucleic acid sequence encoding the CAR of the present invention. According to one embodiment, the nucleic acid molecule encodes amino acid sequence selected from SEQ ID NO: 3, SEQ ID NO: 5 and both SEQ ID NOs: 3 and 5. According to some embodiments, the nucleic acid molecule encodes amino acid sequence SEQ ID NO: 15. According to other embodiment, the nucleic acid comprises nucleic acid sequence SEQ ID NO: 21 or a variant thereof having at least 95% sequence identity to the original sequence. According to certain embodiments, the nucleic acid molecule further encodes amino acid sequence selected from SEQ ID NOs: 17, 18, 19, an analog thereof and any combination thereof. According to some embodiments, the nucleic acid sequence further comprises nucleic acid sequence selected from SEQ ID NO: 22, 23, 24, a variant thereof having at least 95% sequence identity to the original sequence(s), and a combination thereof. According to one embodiment, the nucleic acid molecule of the present invention encodes amino acid sequence selected from SEQ ID NO: 20, SEQ ID NO: 28 and SEQ ID NO: 38. According to another embodiment, the nucleic acid molecule comprises a nucleic acid sequence selected from SEQ ID NO: 25, SEQ ID NO: 29, and a variant thereof having at least 95% sequence identity to the sequence.

According to another aspect, the present invention provides a nucleic acid construct comprising the nucleic acid molecule of the present invention, operably linked to a promoter.

According to a further aspect, the present invention provides a vector comprising the nucleic acid molecule or the nucleic acid construction of the present invention.

According to a certain aspect, the present invention provides a cell comprising the CAR of the present invention or the nucleic acid molecule, nucleic acid construct or the vector of the present invention. According to some embodiments, the cell expresses or is capable of expressing the CAR of the present invention. According to some embodiments, the cell is a lymphocyte. According to some embodiments, the cell is selected from a T cell and a natural killer (NK) cell. According to some embodiments, the present invention provides a T cell expressing the CAR of the present invention.

According to some embodiments, a lymphocyte engineered to express the CAR described herein is provided. According to some embodiments, a T cell engineered to express the CAR described herein is provided. According to additional embodiments, an NK cell engineered to express the CAR described herein is provided.

According to some embodiments, the present invention provides a cell population, comprising a plurality of cells of the present invention, e.g. CAR T-cells.

According to another aspect, the present invention provides a pharmaceutical composition comprising a plurality of cells of the present invention, and a pharmaceutically acceptable carrier. According to some embodiments, the cells are T-cells. According to one embodiment, the present invention provides a pharmaceutical composition comprising a plurality of T-cells expressing or capable of expressing the CAR of the present invention, and a pharmaceutically acceptable carrier. According to some embodiments, the pharmaceutical composition of the present invention is for use in treating cancer. According to some embodiments, the cancer is SLeA positive (SLeA-expressing) cancer. According to some embodiments, the cancer is selected from lung, breast, ovarian, pancreatic, colorectal, stomach, liver, oropharyngeal cancer, head and neck and gallbladder cancer, and squamous cell carcinoma. According to another embodiment, the cancer is selected from lung adenocarcinoma, pancreatic adenocarcinoma, colon adenocarcinoma, Her-2 negative breast carcinoma and pharynx squamous cell carcinoma. According to some embodiments, the pharmaceutical composition is co-administered with another anti-cancer therapy. According to some embodiments, the pharmaceutical composition is formulated for injection or infusion. According to some embodiments, the pharmaceutical composition is formulated for intravenous administration. In certain embodiments, the pharmaceutical composition is formulated for intratumoral administration.

According to yet another aspect, the present invention provides a method for treating cancer in a subject in need thereof comprising administering to the subject a therapeutically effective amount of cells, such as T-cells, of the present invention or a pharmaceutical composition comprising the cells of the present invention.

Any administration mode may be used to deliver the compositions of the present invention to a subject in need thereof, including parenteral and enteral administration modes. According to some embodiments, a composition according to the present invention is administered parenterally.

### BRIEF DESCRIPTION OF DRAWINGS

FIG. 1 shows the affinities (apparent K_{D}) of the native antibody and RA9-23 antibody against the top four binding glycans by glycan microarray, calculated from saturation curves of 16 serial dilutions of antibodies (ranging at 133.3-0.000853 nM).

FIG. 2 shows the binding of the native antibody and RA9-23 antibody to cancer cell lines and their cytotoxicity. RA9-23 clone shows much better binding to human colorectal cancer cell line WiDr (FIG. 2A) and human pancreatic cancer cell line Capan2 (FIG. 2B) in comparison to the native antibody. RA9-23 antibodies show better killing potential compared to the native antibody as examined by complement-dependent cytotoxicity (CDC). Cytotoxicity against WiDr (FIG. 2C) and Capan2 (FIG. 2D) target cells was determined by LDH detection kit (representative of two independent experiments; 2-way ANOVA, *, P<0.05).

FIG. 3 shows a schematic presentation of the constructed CAR comprising RA9-23 scFv.

FIG. 4 shows RA9-23 CAR specificity against the tumor-associated carbohydrate antigen. FIG. 4A shows RA9-23 CAR expression on transduced T cells was evaluated by FACS, using untransduced T cells (UT) as a control. Cells were stained with APC-mouse-anti-human CD3 and FITC-anti-strep-tag or FITC-isotype control. Gated CD3⁺ T cells were plotted showing CAR expression in at least 50% of cells (representative of three independent experiments). FIG. 4B specificity of RA9-23 CAR against its target antigen (SLeA) as evaluated by FACS, in comparison to closely-related glycans (LeA, SLeX), and to irrelevant-N29 CAR (targeting ErbB2) that served as a control. Transduced CAR T cells were stained with biotinylated-polyacrylamide-conjugated glycan antigens (Ag; glycan-PAA-Bio at 1 μM each), then detected with APC-streptavidin (APC-SA). Only RA9-23 CAR T cells were stained, and only with biotinylated-polyacrylamide-conjugated SLeA antigen, but not with other glycan targets demonstrating RA9-23 CAR specificity against SLeA antigen (representative of two independent experiments).

FIG. 5 shows evaluation of off-target cytotoxicity of RA9-23 CAR T cells. FIG. 5A—The ovarian carcinoma cell line OVCAR-8 were stained with RA9-23-hIgG antibody (20 ng/μl) then detected with PE-anti-human IgG-Fc (5 ng/μl) and read by FACS, confirming their expression of SLeA antigen. FIG. 5B—off-target cytotoxicity was evaluated by co-culturing RA9-23 CAR or untransduced T cells with primary human cells (alveolar/pancreatic/cardiac endothelial cells/erythrocytes/Kidney epithelial cells) at 1:2 ratio (E:T), or without target cells (negative control; None), or with SLeA-positive OVCAR-8 cells (positive control), followed by measuring IFN-γ secretion to the growth media by ELISA.

FIG. 6 shows in vitro cytotoxicity of RA9-23 CAR T cells. FIG. 6A—FaDu pharynx squamous cell carcinoma cells were stained with RA9-23-hIgG antibody (20 ng/μl) then detected with PE-anti-human IgG-Fc (5 ng/μl) and read by FACS, confirming expression of SLeA antigen compared to the control (secondary antibody; 2Ab; (representative of three independent experiments). FIG. 6B—RA9-23 CAR T cells or control untransduced T cells (UT) were co-cultured with FaDu cells at 2:1 ratio, then IFN-γ levels in the growth media was measured by ELISA, showing stimulation (IFN-γ secretion) only of RA9-23 CAR-expressing cells (mean±SEM; two tailed unpaired t test, *p<0.032; representative of two independent experiments). (FIG. 6C) To evaluate in vitro cytotoxicity, FaDu target cells (T) were co-cultured for 16 hours with effector (E) RA9-23 CAR T cells or control untransduced T cells (UT) at the indicated E:T ratios, followed by washing (to remove dead cells and effector cells), fixation of remaining target cells with 4% formaldehyde, and nuclei staining with methylene blue. Viability was assessed by reading absorbance of stained cells at 620 nm with Multiskan FC ELISA reader. This analysis clearly showed reduced cell viability with increasing effector RA9-23 CAR T cells, but not with the control cells, demonstrating the cytotoxicity effect of RA9-23 CAR T cells against SLeA-expressing cells (mean±SD; Two-way ANOVA with Bonferroni post-tests, ****p<0.0001; representative of three independent experiments).

FIG. 7 shows in vivo cytotoxicity of RA9-23 CAR T cells after systemic (FIG. 7A) or intratumor (FIG. 7B) administration. Adoptive transfer of RA9-23 CAR T cells significantly inhibited growth of SLeA-expressing FaDu pharynx squamous cell carcinoma tumors. NSG (NOD.Cg-Prkdcscid Il2rgtmlWjl/SzJ) female mice were injected subcutaneously with 0.5×10⁶ FaDu cells. On day 11, mice were irradiated at 2Gy, and on the following day treated by intravenous (systemic) or by intra-tumoral (local) injections of 10⁷ RA9-23 CAR T cells (˜50% transduction) or control untransduced T cells (UT) (n=5 per group). Tumor volume was monitored twice a week. Average fold change of tumor volume was calculated as tumor volume at each time point divided by the baseline tumor volume (day 7 or day 10 for intra-tumoral or intravenous treatments, respectively; mean±SEM; Two-way ANOVA with Bonferroni post-tests, **p<0.0021, ***p<0.0002****p<0.0001; representative of at least two independent experiments).

FIG. 8 shows the specificity of the full-length antibody mutant clone RA9-23 as examined by ELISA inhibition assay against coated SLeA-PAA-Biotin, after pre-incubation of the antibody with specific (SLeA) or non-specific glycans (SLeX and LeA). ****p<0.001.

FIG. 9 shows the specificity of binding of RA9-23 Abs to WiDr cells demonstrated by the treatment of cells with Arthrobacter Ureafaciens Sialidase (AUS) that abrogated binding of RA9-23 IgG to SLeA-expressing WiDr cells (FIG. 9D), in comparison to direct binding of the antibodies (FIG. 9B), their binding to cells treated with heat-inactivated AUS (FIG. 9C) or a secondary antibody (FIG. 9A).

FIG. 10 shows the structures of AcSLeA (FIG. 10A) and closely related glycans: (FIG. 10B—SLeX; FIG. 10C—LeA; FIG. 10D—LeY; FIG. 10E—LeX.

FIG. 11 shows the summary of staining of different types of cancer tissues using RA9-23 indicating for presence of SLeA in lung and pancreatic adenocarcinomas, colon carcinoma and HER2-neg breast carcinoma.

FIG. 12 shows in vitro cytotoxicity of RA9-23 CAR T cells. FIG. 12A shows staining of FaDu pharynx squamous cell carcinoma cells and a collection of primary human cells with RA9-23-hIgG antibody (20 ng/μl), detected with PE-anti-human IgG-Fc (5 ng/μl) and read by FACS. The results confirm expression of SLeA antigen only in FaDu cells compared to the control (unstained or secondary antibody; 2Ab), but in none of the primary human cells. FIG. 12B shows stimulation (IFN-γ expression levels) upon co-culturing FaDu cells or primary human cells with RA9-23 CAR T cells or control untransduced T cells (UT) at 3:1 ratio (E:T; Effector T cells to Target cells ratio), as measured by ELISA.

### DETAILED DESCRIPTION OF THE INVENTION

The present invention discloses a chimeric antigen receptor comprising an antigen binding domain (ABD) that specifically binds to Sialyl Lewis A glycan. The invention is based inter alia on data showing that chimeric antigen receptors comprising such an ABD, and more particularly scFv of RA9-23 antibody, successfully activated T-cells comprising same, and that these T-cells induces cytotoxicity towards cancer cells presenting SLeA (as shown in Examples 5 and 6). Moreover, as shown in Example 8, activation of CAR T-cells specific to SLeA was observed only when they were incubated with cancer cells presenting SLeA but not with normal primary cells. This indicates that treatment with such CAR T-cells is safe and does not induce off-target cytotoxicity.

According to one aspect, the present invention provides a chimeric antigen receptor (CAR) comprising an antigen binding domain that binds specifically to Sialyl Lewis A glycan (SLeA), wherein the antigen binding domain comprises three complementarity determining regions (CDRs) of a heavy-chain variable domain (VH) having amino acid sequence as set forth in SEQ ID NO: 1 and three CDRs of a light-chain variable domain (VL) having amino acid sequence as set forth in SEQ ID NO: 4. According to another embodiment, the present invention provides an analog of said CAR having at least 90% Y⁴ sequence identity to said sequences. According to other embodiments, the analog comprises at least 92%, at least 95% or at least 98% sequence identity to said amino acid sequences. According to some embodiments, the CDR2 of the VH domain (VH-CDR2) comprises amino acid sequence SEQ ID NO: 7. According to some embodiments, the CDR2 of the VH domain (VH-CDR2) comprises amino acid sequence SEQ ID NO: 31. According to some embodiments, CDRs 1, 2, and 3 of the VH domain comprise amino acid sequences SEQ ID NOs: 6, 7 and 8, respectively, and CDRs 1, 2, and 3 of the VL domain comprise amino acid sequences SEQ ID NOs: 9, 10 and 11, respectively. According to other embodiments, the amino acid sequences of CDRs of the VH domain are set forth in SEQ ID NOs: 30, 31 and 8, and the amino acid sequences of CDRs of the VL domain are set forth in SEQ ID NOs: 9, 10, and 11. According to some embodiments, the VH-CDR1 comprises amino acid sequence selected from SEQ ID NOs: 6 and 30. According to other embodiments, the VH-CDR1 comprises amino acid sequence selected from SEQ ID NOs: 7 and 31. Thus, according to one embodiment, the present invention provides a chimeric antigen receptor (CAR) comprising an antigen binding domain that binds specifically to Sialyl Lewis A glycan (SLeA), wherein the antigen binding domain comprises a VH and VL domains, each comprising 3 CDRs, wherein CDR 1, 2, and 3 of the VH domain comprise amino acid sequences SEQ ID NOs: 6, 7 and 8, respectively, and CDR 1, 2, and 3 of the VL domain comprise amino acid sequences SEQ ID NOs: 9, 10 and 11, respectively. According to some embodiments, the present invention provides a chimeric antigen receptor (CAR) comprising an antigen binding domain that binds specifically to Sialyl Lewis A glycan (SLeA), wherein the antigen binding domain comprises a VH and VL domains, each comprising 3 CDRs, wherein the amino acid sequences of CDRs of the VH domain are set forth in SEQ ID NOs: 30, 31 and 8, and the amino acid sequences of CDRs of the VL domain are set forth in SEQ ID NOs: 9, 10, and 11. According to other embodiments, the present invention provides a CAR comprising an ABD that binds specifically to Sialyl Lewis A glycan (SLeA), wherein the ABD comprises a VH and VL domains each comprising three CDRs and four FRs, wherein the VH-CDR 1, 2 and 3 comprise amino acid sequences SEQ ID NOs: 30, 31, and 8, respectively, the VL-CDRs 1, 2 and 3 comprise amino acid sequences SEQ ID NOs: 9, 10 and 11, respectively, VH-FRs 1, 2 and 4 comprises acid sequences SEQ ID NOs: 39, 42 and 43, respectively, and the VL-FR 1 comprises acid sequences SEQ ID NO: 44.

The terms “chimeric antigen receptor” or “CAR” are used herein interchangeably and refer to engineered recombinant polypeptide or receptor which are grafted onto cells and comprises at least (1) an extracellular domain comprising an antigen-binding region, e.g., a single chain variable fragment of an antibody or a whole antibody, (2) a transmembrane domain to anchor the CAR into a cell, and (3) one or more cytoplasmic signaling domains (also referred to herein as “an intracellular signaling domains”). The extracellular domain comprises an antigen binding domain (ABD) and optionally a spacer or hinge region. The antigen binding domain of the CAR targets a specific antigen. The targeting regions may comprise full length heavy chain, Fab fragments, or single chain variable fragment (scFvs).

The terms “antigen binding portion”, “antigen binding domain” and “ABD” refer to one or more fragments of an antibody that retain the ability to specifically bind to an antigen. It has been shown that the antigen binding function of an antibody can be performed by fragments of a full-length antibody. Such ABD may also be bispecific, dual specific, or multi-specific formats; specifically binding to two or more different antigens. Examples of binding fragments encompassed within the term “antigen binding portion” include (i) a Fab fragment, a monovalent fragment consisting of the VL, VH, CL and CH1 domains; (ii) a F(ab′)₂fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb, which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). Such single chain antibodies are also intended to be encompassed within the term “antigen binding portion”. In certain embodiments of the invention, scFv molecules are incorporated into a fusion protein. Other forms of single chain antibodies, such as diabodies are also encompassed. The antigen binding domain can be derived from the same species or a different species for or in which the CAR will be used. In one embodiment, the antigen binding domain is a scFv.

The terms “light chain variable region”, “VL” and “V_{L}” are used herein interchangeably and refer to a light chain variable region of an antibody capable of binding to SLeA glycan. The terms “heavy chain variable region”, “VH” and “V_{H}” are used herein interchangeably and refer to a heavy chain variable region of an antibody capable of binding to SLeA glycan.

According to any one of the above embodiments, the VL and VH domains in the scFv may be in any order, such as N′-VH-VL-C′ or N′-VL-VH-C′. The VH and VL domains may be linked by a linker.

According to some embodiments, the extracellular domain comprises a hinge region. The extracellular spacer or hinge region of a CAR is located between the antigen binding domain and a transmembrane domain. Extracellular spacer domains may include, but are not limited to, Fc fragments of antibodies or fragments or derivatives thereof, hinge regions of antibodies or fragments or derivatives thereof, constant domains such as CH2 region or CH3 region of antibodies, accessory proteins, artificial spacer sequences or combinations thereof.

As used herein, the term “CDR” refers to the complementarity determining region within antibody variable sequences. There are three CDRs in each of the variable regions of the heavy chain and the light chain, which are designated CDR1, CDR2 and CDR3 (or specifically VH CDR1, VH CDR2, VH CDR3, VL CDR1, VL CDR2, and VL CDR3), for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. Still other CDR boundary definitions may not strictly follow one of known systems, but will nonetheless overlap with the Kabat CDRs, although they may be shortened or lengthened in light of prediction or experimental findings that particular residues or groups of residues or even entire CDRs do not significantly impact antigen binding. Determination of CDR sequences from antibody heavy and light chain variable regions can be made according to any method known in the art, including but not limited to the methods known as KABAT, Chothia and IMGT. The selected set of CDRs may include sequences identified by more than one method, namely, some CDR sequences may be determined using KABAT and some using IMGT. According to one embodiments, the CDRs are defined using KABAT method

As used herein, the terms “framework”, “framework domain”, “framework region” or “framework sequence” refer to the remaining sequences of a variable region minus the CDRs. Because the exact definition of a CDR sequence can be determined by different systems, the meaning of a framework sequence is subject to correspondingly different interpretations. The six CDRs also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represent two or more of the four sub-regions constituting a framework region.

The term “transmembrane domain” refers to the region of the CAR, which crosses or bridges the plasma membrane. The transmembrane domain of the CAR of the invention is the transmembrane region of a transmembrane protein, an artificial hydrophobic sequence or a combination thereof. According to some embodiments, the term comprises also transmembrane domain together with extracellular spacer or hinge region.

The term “intracellular domain” refers to the intracellular part of the CAR and may be an intracellular domain of T cell receptor or of any other receptor (e.g., TNFR superfamily member) or portion thereof, such as an intracellular activation domain (e.g., an immunoreceptor tyrosine-based activation motif(ITAM)-containing T cell activating motif), an intracellular costimulatory domain, or both.

The terms “binds specifically” or “specific for” with respect to an antigen-binding domain of an antibody or of a fragment thereof refers to an antigen-binding domain which recognizes and binds to a specific antigen, but does not substantially recognize or bind other molecules in a sample. The term encompasses that the antigen-binding domain binds to its antigen with high affinity and binds other antigens with low affinity. An antigen-binding domain that binds specifically to an antigen from one species may bind also to that antigen from another species. This cross-species reactivity is not contrary to the definition of that antigen-binding domain as specific. The term “K_{D}”, as used herein, is intended to refer to the dissociation constant of a particular antibody-antigen interaction. K_{D} is calculated by kₐ/k_{d}. The term “kₒₙ” or “kₐ”, as used herein, is intended to refer to the on rate constant for association of an antibody to the antigen to form the antibody/antigen complex. The term “k_{off}” or “k_{d}”, as used herein, is intended to refer to the off rate constant for dissociation of an antibody from the antibody/antigen complex.

The terms “Sialyl Lewis A glycan”, “SLe^{a}” and “SLeA” are used herein interchangeably and refer to Siaα2-3Galβ1-3[Fuca-4]GlcNAc tetrasaccharide carbohydrate also known as antigen 19-9 (CA19-9), and having the structure as presented in structure I and schematically presented in Scheme I. This tetrasaccharide can be conjugated to different underlying structures such as carbohydrate/s, protein, lipid, synthetic linker/s or scaffolds.

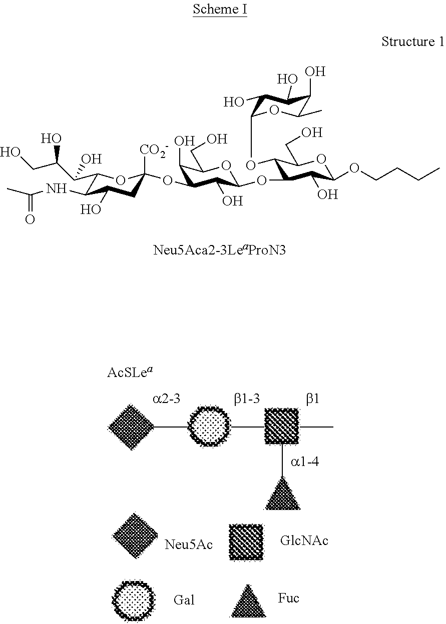

According to some embodiments, the ABD is a scFv, wherein the VH domain comprises amino acid sequence SEQ ID NO: 1 and the VL domain comprises amino acid sequence SEQ ID NO: 4. According to some embodiments, the spacer comprises amino acid sequence comprising from 1 to 10 repetitions of amino acid sequence SEQ ID NO: 16. According to some embodiments, the spacer comprises 2, 3, 4, 5, or 6 repetitions of amino acid sequence SEQ ID NO: 16. According to one embodiment, the spacer comprises amino acid sequence comprising 3 repetitions of amino acid sequence SEQ ID NO: 16. According to some embodiment, the present invention provides a CAR comprising amino acid sequence SEQ ID NO: 37. According to some embodiments, the CAR of the present invention comprises amino acid sequence SEQ ID NO: 38.

According to any one of the above embodiments, the CDR2 of the VH domain (VH-CDR2) comprises at least one non-conservative substitution. Thus, according to some embodiments, the present invention provides a chimeric antigen receptor (CAR) comprising an antigen binding domain that binds specifically to Sialyl Lewis A glycan (SLeA), wherein the antigen binding domain comprises three complementarity determining regions (CDRs) of a heavy-chain variable domain (VH) having amino acid sequence as set forth in SEQ ID NO: 1 and three CDRs of a light-chain variable domain (VL) having amino acid sequence as set forth in SEQ ID NO: 4, wherein the VH-CDR2 comprises at least one non-conservative substitution. According to one embodiment, the native VH-CDR2 comprises amino acid sequence SEQ ID NO: 7 and further comprising at least one non-conservative substitution. According to one embodiment, the native VH-CDR2 comprises amino acid sequence SEQ ID NO: 7 in which one or more amino acids are substituted by a non-conservative substitution. According to one embodiment, the native VH-CDR2 comprises amino acid sequence SEQ ID NO: 31 and further comprising at least one non-conservative substitution.

The term “non-conservative substitutions”, as used with respect to an amino acid sequence shall mean the substitution of one amino acid by another which has different properties (i.e., charge, polarity, hydrophobicity, structure). Examples of the non-conservative substitution include a substitution of a hydrophobic residue such as isoleucine, valine, leucine, alanine, phenylalanine, tyrosine, tryptophan or methionine for a polar or charged amino acid residue such as lysine, arginine, glutamine, asparagine, aspartate, glutamate, histidine serine, threonine, or cysteine. Likewise, the present disclosure contemplates the substitution of a charged amino acid such as lysine, arginine, histidine, aspartate and glutamate for an uncharged residue including, but not limited to serine, threonine, asparagine, glutamine, or glycine. In certain embodiments, non-conservative substitutions include substitution of an uncharged, hydrophobic amino acid such as leucine with a charged amino acid such as aspartic acid, lysine, arginine, or glutamate.

According to some embodiments, the substitution in VH-CDR2 is at position 61 of SEQ ID NO:1 for an amino acid selected from Asn and Gln. According to one embodiment, the VH-CDR2 has amino acid sequence SEQ ID NO: 12. According to one embodiment, the CAR comprises an ABD comprising VH-CDR2 having amino acid sequence SEQ ID NO: 12. According to one embodiment, the VH CDR2 has an amino acid sequence SEQ ID NO: 36.

According to one embodiment, the present invention provides a CAR comprising an antigen binding domain that binds specifically to Sialyl Lewis A glycan (SLeA), wherein the CDR1, CDR2 and CDR3 of the VH domain have amino acid sequences SEQ ID NOs: 6, 12 and 8, respectively. According to another embodiment, the CDR1, CDR2 and CDR3 of the VL domain have amino acid sequences SEQ ID NOs: 9, 10 and 11, respectively. According to yet another embodiment, CDRs 1, 2, and 3 of the VH domain comprise amino acid sequences SEQ ID NOs: 6, 12 and 8, respectively, and CDRs 1, 2, and 3 of the VL domain comprise amino acid sequences SEQ ID NOs: 9, 10 and 11, respectively. According to some embodiments, the amino acid sequences of CDRs of the VH domain are set forth in SEQ ID NOs: 30, 36 and 8, and the amino acid sequences of CDRs of the VL domain are set forth in SEQ ID NOs: 9, 10, and 11. Thus, according to some embodiments, the present invention provides a chimeric antigen receptor (CAR) comprising an antigen binding domain that binds specifically to Sialyl Lewis A glycan (SLeA), wherein the antigen binding domain comprises a heavy-chain variable domain (VH) comprising three complementarity determining regions (CDRs) having amino acid sequences SEQ ID NOs: 6, 12 and 8, and a light-chain variable domain (VL) comprising three CDRs having amino acid sequences SEQ ID NOs: 9, 10 and 11. According to some embodiments, the present invention provides a chimeric antigen receptor (CAR) comprising an antigen binding domain that binds specifically to Sialyl Lewis A glycan (SLeA), wherein the antigen binding domain comprises a heavy-chain variable domain (VH) comprising three complementarity determining regions (CDRs) having amino acid sequences SEQ ID NOs: 30, 36 and 8 and the CDR1, CDR2 and CDR3 of the VL domain have amino acid sequences SEQ ID NOs: 9, 10, and 11.

According to any one of the above embodiments, the ABD of the CAR of the present invention comprises at least 1 non-conservative substitution in the framework sequence(s) of the VH domain and/or of VL domain. According to one embodiment, the ABD comprises at least 1 non-conservative substitution at the framework sequences of the VH domain. According to another embodiment, the ABD further comprises at least 1 non-conservative substitution at framework sequences of the VL domain. According to other embodiments, the ABD comprises at least 2 non-conservative substitutions in the framework sequences of the variable region. According to other embodiments, the ABD comprises at least 2 non-conservative substitutions in framework sequences of the VH domain, of the VL domain or of both VH and VL domains. According to other embodiments, the ABD comprises at least 3 non-conservative substitutions in the framework sequences. According to other embodiments, the ABD comprises at least 4 non-conservative substitutions in the framework sequences. According to other embodiments, the ABD comprises 5, 6, 7 or 8 non-conservative substitutions in the framework sequences of either VH, VL or both VH and VL domains. According to some embodiments, the ABD comprises from 2 to 5 or from 3 to 4 non-conservative substitutions in the framework sequences. According to some embodiments, at least 2 of said non-conservative substitutions is substitution for proline amino acid residue. According to some embodiments, the substitution(s) is in VH-FR1. According to other embodiments, the substitution(s) is in VH-FR4. According to yet another embodiment, the substitution(s) is in VL-FR1. According to some embodiments, the substitutions are in VH-FR1, VH-FR4 and VL-FR1.

According to one embodiment, the ABD comprises non-conservative substitutions in at least one position of the positions selected from positions 1, 110, 114 of SEQ ID NO: 1 or 2, position 22 of SEQ ID NO: 4 and any combination thereof.

According to one embodiment, the substitution of the amino acid in position 1 of SEQ ID NO: 1 or 2 is a substitution for a positively charged amino acid residue. According to one embodiment, the positively charged amino acid residue is selected from Lys and Arg.

According to one embodiment, the ABD comprises non-conservative substitutions at positions 110, 114 or both of SEQ ID NO: 1 or 2, wherein the substitution is for proline.

According to yet another embodiment the ABD comprises non-conservative substitutions at position 22 of SEQ ID NO: 4, wherein the substitution is for proline.

According to some embodiments, the present invention provides a CAR comprising an ABD that binds specifically to Sialyl Lewis A glycan (SLeA), wherein the ABD comprises a VH and VL domains each comprising three CDRs and four FRs, wherein the VH-CDR 1, 2 and 3 comprise amino acid sequences SEQ ID NOs: 30, 12, and 8, respectively, the VL-CDRs 1, 2 and 3 comprise amino acid sequences SEQ ID NOs: 9, 10 and 11, respectively, VH-FRs 1, 2 and 4 comprises acid sequences SEQ ID NOs: 39, 42 and 43, respectively, and the VL-FR 1 comprises acid sequences SEQ ID NO: 44. According to some embodiments, the VH-CDR 1 comprises an amino acid sequence selected from SEQ ID NOs: 30 and 6 and the VH-CDR2 comprises an amino acid sequence selected from SEQ ID NO: 12 and 36.

According to some embodiments, VH-CDR 1 and 2 comprise amino acid sequences SEQ ID NOs: 30 and 36, respectively and the VH-FRs 1 and 2 comprise amino acid sequences SEQ ID NOs: 40 and 42, respectively.

According to some embodiments, the VH-FR3 comprises amino acid sequence SEQ ID NO: 45. According to one embodiment, the VL-FR2 comprises amino acid sequence SEQ ID NO: 46. According to another embodiment, the VL-FR3 comprises amino acid sequence SEQ ID NO: 47. According to certain embodiments, the VL-FR4 comprises amino acid sequence SEQ ID NO: 48.

Exemplary sequences of CDRs and FRs are provided in Tables 1 and 2, respectively.

TABLE 1
Exemplary CDR sequences

| | ID | |
|---|---|---|
| Name | number | Sequence |
| Set 1 VH-CDR 1 | 6 | GFTFSDAWMD |
| Set 2 VH-CDR 1 | 30 | DAWMD |
| Set 1 VH-CDR 2 | 7 | NKGNNHATYYAESVKG |
| Set 1 VH-CDR 2-mutated | 12 | NKGNNHATNYAESVKG |
| Set 2 VH-CDR 2 | 31 | EIGNKGN |
| Set 2 VH-CDR 2-mutated | 36 | EIGNKGNNHATNYAESVKG |
| VH-CDR 3 | 8 | RFAY |
| VL-CDR 1 | 9 | KASQDIN |
| VL-CDR 2 | 10 | RANRLVD |
| VL-CDR 3 | 11 | LQYDEFP |

TABLE 2
Exemplary framework domain sequences

| | ID | |
|---|---|---|
| Name | number | Sequence |
| Set 1 VH-FR1 | 39 | KVKLEESGGGLVQPGGSMKLSCAAS |
| Set 2 VH-FR1 | 40 | KVKLEESGGGLVQPGGSMKLSCAASGFTFS |
| Set 1 VH-FR2 | 41 | WVRQSPEKGLEWVAEIG |
| Set 2 VH-FR2 | 42 | WVRQSPEKGLEWVA |
| VH-FR3 | 45 | RFTVSRDDSKSRVYLQMNSLRVEDTGTYYC |
| | | TT |
| VH-FR4 | 43 | WGQGTPVTVPA |
| VL-FR1 | 44 | DIKMTQSPSSMYASLGERVTIPC |
| VL-FR2 | 46 | WFQQKPGKSPKTLIY |
| VL-FR3 | 47 | GVPSRFSGSGSGQDYSLTISSLEYEDMGIY |
| | | YC |
| VL-FR4 | 48 | GGGTKLEIK |

According to one embodiment, the CDRs 1, 2, and 3 of the VH domain comprises amino acid sequences SEQ ID NOs: 30, 36 and 8, respectively, the CDRs 1, 2, and 3 of the VL domain comprise amino acid sequences SEQ ID NOs: 9, 10 and 11, the VH-FRs 1, 2 and 4 comprise amino acid sequences SEQ ID NOs: 40, 42 and 43, respectively, and the VL-FR1 comprises acid sequences SEQ ID NO: 44. According to another embodiment, the CDRs 1, 2, and 3 of the VH domain consist of amino acid sequences SEQ ID NOs: 30, 36 and 8, respectively, the CDRs 1, 2, and 3 of the VL domain consist of amino acid sequences SEQ ID NOs: 9, 10 and 11, the VH-FRs 1, 2 and 4 comprise amino acid sequences SEQ ID NOs: 40, 42 and 43, respectively, and the VL-FR1 comprises acid sequences SEQ ID NO: 44. According to one embodiment, the CDRs 1, 2, and 3 of the VH domain consist of amino acid sequences SEQ ID NOs: 30, 36 and 8, respectively, the CDRs 1, 2, and 3 of the VL domain consist of amino acid sequences SEQ ID NOs: 9, 10 and 11, the VH-FRs 1, 2 and 4 consist of amino acid sequences SEQ ID NOs: 40, 42 and 43, respectively, and the VL-FR1 consist of amino acid sequences SEQ ID NO: 44. According to some embodiments, CDRs 1, 2, and 3 of the VH domain comprise amino acid sequences SEQ ID NOs: 30, 36 and 8, respectively, CDRs 1, 2, and 3 of the VL domain comprise amino acid sequences SEQ ID NOs: 9, 10 and 11, respectively, VH-FRs 1, 2, 3 and 4 comprise amino acid sequences SEQ ID NOs: 40, 42, 45 and 43, respectively, and the VL-FR1, 2, 3 and 4 comprise amino acid sequences SEQ ID NOs: 44, 46, 47 and 48, respectively.

According to one embodiment, the VH domain of the ABD comprises amino acid sequence SEQ ID NO: 3. According to another embodiment, the VL domain of the ABD of the CAR of the present invention comprises amino acid sequence SEQ ID NO: 5. According to yet another embodiment, the present invention provides a CAR comprising an ABD, wherein the ABD comprises VH domain comprising amino acid sequence SEQ ID NO: 3 and a VL domain comprising amino acid sequence SEQ ID NO: 5. According to further embodiments, the present invention provides a CAR comprising an ABD comprising VH domain having amino acid sequence SEQ ID NO: 3 and a VL domain having amino acid sequence SEQ ID NO: 5.

According to any one of the aspects and embodiments of the invention, when referring to CAR or ABD the terms “comprising the amino acid sequence set forth in SEQ ID NO: X”, “comprising SEQ ID NO: X” and “having SEQ ID NO: X” are used herein interchangeably. The terms “consisting of the amino acid sequence set forth in SEQ ID NO: X”, “consisting of SEQ ID NO: X” and “of SEQ ID NO: X” are used herein interchangeably.

The same rule holds for nucleic acid sequence. Thus the terms “nucleic acid comprising the nucleic acid sequence set forth in SEQ ID NO: X”, “nucleic acid comprising SEQ ID NO: X” and “nucleic acid having SEQ ID NO: X” are used herein interchangeably. The terms “nucleic acid consisting of the nucleic acid sequence set forth in SEQ ID NO: X”, “nucleic acid consisting of SEQ ID NO: X” and “nucleic acid of SEQ ID NO: X” are used herein interchangeably.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” also encompasses the meaning of “consisting of” and “consisting essentially of”, and may be substituted by these terms. Thus, according to any aspect or embodiment of the present invention, the statement such as VH or VL comprising amino acid sequence X encompasses also the meaning that the VH or VL consisting of amino acid sequence X.

Thus, according to some embodiments, the present invention provides a CAR comprising an ABD comprising a VH domain consisting of amino acid sequence SEQ ID NO: 3. According to another embodiment, the VL domain of the ABD of the present invention consists of amino acid sequence SEQ ID NO: 5. According to yet another embodiment, the present invention provides a CAR comprising an ABD comprising VH domain consisting of amino acid sequence SEQ ID NO: 3 and a VL domain consisting of amino acid sequence SEQ ID NO: 5.

According to any one of the above embodiments, the ABD of the present invention further comprises at least one conservative substitution in the framework(s) of the VH domain, VL domain or both, i.e. being a conservative analog of the ABD of the present invention. According to one embodiment, the substitution is not at positions 1, 110, 114 of SEQ ID NO: 1 or 2 and not at position 22 of SEQ ID NO: 4. According to any one of the above embodiments, the ABD of the present invention further comprising at least one conservative substitution in the framework(s) of the VH domain wherein the resulted VH domain has at least 90% sequence identity to SEQ ID NO: 3 and the VL comprises amino acid sequence SEQ ID NO: 5. According to one embodiment, the ABD of the present invention further comprises at least one conservative substitution in the framework(s) of the VL domain, wherein the resulted VL domain has at least 90% sequence identity to SEQ ID NO: 5 and the VH comprises amino acid sequence SEQ ID NO: 3. According to a further embodiment, the present invention provides a CAR comprising an ABD comprising at least one conservative substitution in the framework(s) of the VH and of the VL domains, wherein the resulted VH domain has at least 90% sequence identity to SEQ ID NO: 3 and the resulted VL domain has at least 90% sequence identity to SEQ ID NO: 5. According to some embodiments, the VH domain comprising such conservative substitution(s) has at least 91%, at least 92%, at least 93%, at least 94%, at least 95%, at least 96%, at least 97%, at least 98%, at least 99% sequence identity to SEQ ID NO: 3. According to other embodiments, the VL domain comprising such conservative substitution(s) has at least 91%, at least 92%, at least 93%, at least 94%, at least 95%, at least 96%, at least 97%, at least 98%, at least 99% sequence identity to SEQ ID NO: 5.

The term “conservative substitution” as used herein denotes the replacement of an amino acid residue by another, without altering the overall conformation and biological activity of the peptide, polypeptide or protein, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, according to one table known in the art, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

According to some embodiments, the VH and the VL domains of the ABD of the CAR of the present invention are linked by a spacer to form a single chain variable fragment (scFv). The terms “linker” or “spacer” in the context of CAR relates to any peptide capable of connecting two domains of the CAR or two distinguishable sections of the CAR such as variable domains with its length depending on the kinds of variable domains to be connected. According to some embodiments, the spacer comprises amino acid sequence comprising from 1 to 10 repetitions of amino acid sequence SEQ ID NO: 16. According to some embodiments, the spacer comprises 2, 3, 4, 5, or 6 repetitions of amino acid sequence SEQ ID NO: 16. According to one embodiment, the spacer comprises amino acid sequence comprising 3 repetitions of amino acid sequence SEQ ID NO: 16. According to some embodiments, the antigen binding domain of the CAR according to the present invention comprises amino acid sequence SEQ ID NO: 15. According to another embodiment, the scFv comprises amino acid sequence analog of SEQ ID NO: 15 having at least 90% sequence identity to SEQ ID NO: 15, wherein the amino acid alteration(s) is not at positions corresponding to positions 1, 110, 114 of SEQ ID NO: 3 and not at positions corresponding to positions 22 of SEQ ID NO: 5.

The term “analog” refers polypeptide, peptide or protein which differs by one or more amino acid alterations (e.g., substitutions, additions or deletions of amino acid residues) from the original sequence, having at least 70% sequence identity to the original sequence and still maintains the properties of the parent polypeptide, peptide or protein. According to one embodiment, the analog comprises at least one modification selected from a substitution, deletion and addition. According to some embodiments, the modification is a substitution. According to some embodiments, the peptide analog has at least 80%, at least 90% or at least 95% sequence identity to the original peptide. According to one embodiment, the analog has about 70% to about 95%, about 80% to about 90% or about 85% to about 95% sequence identity to the original peptide. According to some embodiments, the analog of the present invention comprises the sequence of the original peptide in which 1, 2, 3, 4, or 5 substitutions were made. According to one embodiment, the substitution is a conservative substitution.

The CAR of the present invention comprises a transmembrane domain (TM domain), one or more costimulatory domains and an activation domain.

In one embodiment of the invention, the CAR includes a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154 on an analog thereof. According to one embodiment, the TM domain is a TM domain of a receptor selected from CD28 and CD8, or an analog thereof having at least 85% amino acid identity to the original sequence.

In some embodiments of the invention, the CAR comprises a costimulatory domain, e.g., a costimulatory domain comprising a functional signaling domain of a protein selected from the group consisting of OX40, CD2, CD27, CD28, CD5, ICAM-1, LFA-1 (CD11a/CD18), ICOS (CD278), 4-1BB (CD137), an analog thereof and a combination thereof. According to one embodiment, the costimulatory domain is selected from a costimulatory domain of a protein selected from CD28, 4-1BB, OX40, an analog thereof having at least 85% amino acid identity to the original sequence, and any combination thereof. According to some embodiments, the CAR of the present invention comprises two or more costimulatory domains. According to one embodiment, the CAR comprises costimulatory domains of CD28 and 4-1BB.

According to one embodiment, the TM domain and the costimulatory domain of the CAR are both derived from CD28. According to one embodiment, the TM domain and the costimulatory domain have amino acid sequence SEQ ID NO: 17. According to another embodiment, the TM domain and the costimulatory domain have an amino acid sequence which is an analog of SEQ ID NO: 17 having at least 85% amino acid identity to SEQ ID NO: 17.

According to some embodiments, the antigen binding domain is linked to the TM domain via a spacer. According to one embodiment, the spacer comprises amino acid sequence comprising from 1 to 6 repetitions, such as 1, 2, 3, 4, 5 or 6 repetitions, of amino acid sequence SEQ ID NO: 16. According to one embodiment, the spacer comprises amino acid sequence comprising 2 repetitions of amino acid sequence SEQ ID NO: 16.

According to any one of the above embodiments, the CAR comprises an activation domain selected from FcRγ (gamma) and CD3-ζ (CD3-zetta) activation domains, or any other sequence that contains an intracellular tyrosine activating motif (ITAM). According to one embodiment, the activation domain is FcRγ domain. According to one embodiment, FcRγ domain has amino acid sequence SEQ ID NO: 18 or an analog thereof having at least 85% amino acid identity to the original sequence.

The term “CD28” refers to cluster of differentiation 28 protein. In some embodiments, the CD28 is a human CD28.

The term “CD8” refers to cluster of differentiation 8 protein being a transmembrane glycoprotein and serving as a co-receptor for the T cell receptor. According to one embodiment, the CD8 is a human CD8.

The terms “ICOS” and “Inducible T-cell COStimulator” refer to CD278 which is a CD28-superfamily costimulatory molecule. According to one embodiment, the ICOS is a human ICOS.

The term “4-1BB” refers to a CD137 protein which is a member of the tumor necrosis factor receptor family and has costimulatory activity for activated T cells. According to one embodiment, 4-1BB is a human 4-1BB.

The terms “CD3ζ” and “CD3-zetta” refer to a ζ (zetta) chain of CD3 (cluster of differentiation 3) T cell co-receptor participating in activation of both the cytotoxic and helper T cells. According to one embodiment, CD3ζ comprises an immunoreceptor tyrosine-based activation motif (ITAM). According to one embodiment, the CD3ζ is human CD3ζ. CD3 is sometimes also referred as CD247.

The term “FcRγ” refers to Fc gamma receptors, which generate signals within their cells through ITAM. These are immunoglobulin superfamily receptors that are found on various innate as well as adaptive immune cells, where the extracellular part binds IgGs the activation signal is transduced through two ITAMs located on its cytoplasmic tail.

According to any one of the above embodiments, the CAR further comprises a leading peptide. According to one embodiment, the leading peptide is located N-terminally to the ABD. According to one embodiment, the leading peptide has amino acid sequence SEQ ID NO: 19 or an analog thereof having at least 85% amino acid identity.

The term “leader peptide”, “leading peptide”, “lead peptide”, “signaling peptide” and “signal peptide” are used herein interchangeably and refer to a peptide that translocates or prompts translocation of the target protein to cellular membrane.

According to any one of the above embodiments, the CAR of the present invention further comprises a tag sequence. The term “tag” or “label” refers to a moiety which is attached, conjugated, linked or bound to, or associated with, a compound (for example a protein, peptide, amino acid, nucleic acid and/or carbohydrate) and which may be used as a means of, for example, identifying, detecting and/or purifying a compound. According to some embodiments, the tag is selected haemagglutinin tag, myc tag, poly-histidine tag, protein A, glutathione S transferase, Glu-Glu affinity tag, substance P, FLAG peptide, streptavidin (strep) binding peptide and human FC tag. According to some embodiments, the tags is a strep-tag. According to one embodiment, the tag has amino acid sequence SEQ ID NO: 26.

According to some embodiments, the present invention provides a CAR comprising a scFv comprising an antigen binding domain that binds specifically SLeA, a TM selected from the TM of CD8 and CD28, a costimulatory domain selected from a costimulatory domain of a protein selected from the group consisting of OX40, CD28, 4-1BB (CD137), and combinations thereof, and an activation domain selected from FcRγ and CD3-ζ activation domains. According to some embodiments, the CAR comprises an scFv comprising an amino acid sequence selected from SEQ ID NO: 15 and 37, the TM of CD28, a costimulatory domain of CD28, 4-1BB or both, and an activation domain of FcRγ. According to some embodiments, the scFv comprises an analog of an amino acid sequence selected from SEQ ID NO: 15 and 37 having at least 90% to said sequences.

According to one embodiment, the present invention provides a CAR comprising amino acid sequence SEQ ID NO: 20. According to some embodiments, the present invention provides a CAR comprising amino acid sequence analog of SEQ ID NO: 20 having at least 90% sequence identity to SEQ ID NO: 20. According to another embodiment, the ABD comprises amino acid sequence analog of SEQ ID NO: 20, wherein the amino acid alteration(s) is not at positions corresponding to positions 1, 110, 114 of SEQ ID NO: 3 and not at positions corresponding to positions 22 of SEQ ID NO: 5. According to one embodiment, the present invention provides a CAR consisting of amino acid sequence SEQ ID NO: 20.

According to one embodiment, the present invention provides a CAR comprising amino acid sequence SEQ ID NO: 28. According to some embodiments, the present invention provides a CAR comprising amino acid sequence analog of SEQ ID NO: 28 having at least 90% sequence identity to SEQ ID NO: 28. According to another embodiment, the ABD comprises amino acid sequence analog of SEQ, wherein the amino acid alteration(s) is not at positions corresponding to positions 1, 110, 114 of SEQ ID NO: 3 and not at positions corresponding to positions 22 of SEQ ID NO: 5. According to one embodiment, the present invention provides a CAR consisting of amino acid sequence SEQ ID NO: 28.

According to one embodiment, the present invention provides a CAR comprising amino acid sequence SEQ ID NO: 38. According to some embodiments, the present invention provides a CAR comprising amino acid sequence analog of SEQ ID NO: 38 having at least 90% sequence identity to SEQ ID NO: 38. According to one embodiment, the present invention provides a CAR consisting of amino acid sequence SEQ ID NO: 38.

According to any one of the above embodiments, the CAR of the present invention is capable of activating or activates T cells. According to one embodiment, the CAR of the present invention is capable of promoting T cell proliferation, generation and/or survival. According to some embodiments, the T-cells are selected from memory, regulatory, helper and natural killer T-cells. As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. Activation results is clonal expansion of T cells, upregulation of activation markers on the cell surface, differentiation into effector cells, induction of cytotoxicity or cytokine secretion, induction of apoptosis, or a combination thereof. As used herein, “improving cell survival” and “promoting cell survival” refers to an increase in the number of cells that survive a given condition or period, as compared to a control, e.g., the number of cells that would survive the same conditions in the absence of treatment. Conditions can be in vitro, in vivo, ex vivo, or in situ. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition.

According to another aspect, the present invention provides a nucleic acid molecule encoding the CAR according to any one of the above embodiments and aspects. All aspects and embodiments defined above apply herein as well. Thus, according to one embodiment, the present invention provides a nucleic acid molecule encoding a chimeric antigen receptor (CAR) comprising an antigen binding domain that binds specifically to Sialyl Lewis A glycan (SLeA), wherein the antigen binding domain comprises three complementarity determining regions (CDRs) of a heavy-chain variable domain (VH) having amino acid sequence as set forth in SEQ ID NO: 1 and three CDRs of a light-chain variable domain (VL) having amino acid sequence as set forth in SEQ ID NO: 4. According to some embodiments, the ABD of the CAR comprises VH and VL domains, wherein CDRs 1, 2, and 3 of the VH domain comprise amino acid sequences SEQ ID NOs: 6, 7 and 8, respectively, and CDRs 1, 2, and 3 of the VL domain comprise amino acid sequences SEQ ID NOs: 9, 10 and 11, respectively. According to other embodiments, the ABD of the CAR comprises VH and VL domains, wherein CDRs 1, 2, and 3 of the VH domain comprise amino acid sequences SEQ ID NOs: 30, 31, and 8, respectively, and CDRs 1, 2, and 3 of the VL domain comprise amino acid sequences SEQ ID NOs: 9, 10 and 11, respectively. According to another embodiment, the VH-CDR2 comprises at least one non-conservative substitution. According to another embodiment, VH-CDRs 2 comprise amino acid sequences selected from SEQ ID NOs: 12 and 36.

According to some embodiments, the nucleic acid molecule encodes at least one chain of the CAR comprising a VH domain comprising amino acid sequence SEQ ID NO: 1 and a VL domain comprising amino acid sequence SEQ ID NO: 4. According to one embodiment, the nucleic acid molecule encodes scFv comprising VH domain comprising amino acid sequence SEQ ID NO: 1 and VL domain comprising amino acid sequence SEQ ID NO: 4. According to one embodiment, the nucleic acid molecule encodes both SEQ ID NO: 1 and SEQ ID NO: 4.

According to another embodiment, the nucleic acid molecule encodes at least one chain of the CAR comprising a VH domain comprising amino acid sequence SEQ ID NO: 3 and a VL domain comprising amino acid sequence SEQ ID NO: 5. According to one embodiment, the nucleic acid molecule encodes scFv comprising VH domain comprising amino acid sequence SEQ ID NO: 3 and VL domain comprising amino acid sequence SEQ ID NO: 5. According to one embodiment, the nucleic acid molecule encodes both SEQ ID NO: 3 and SEQ ID NO: 5.

According to one embodiment, the nucleic acid molecule comprises nucleic acid sequence SEQ ID NOs: 13 or a variant thereof having at least 95% sequence identity to the original sequence. According to another embodiment, the nucleic acid molecule comprises comprising nucleic acid sequence SEQ ID NOs: 14 or a variant thereof having at least 95% sequence identity to the original sequence. According to a further embodiment, the nucleic acid molecule comprises nucleic acid sequences SEQ ID NOs: 13 and 14 or a variant thereof having at least 95% sequence identity to the original sequence.

The terms “homolog” “variant”, “DNA variant”, “sequence variant” and “polynucleotide variant” are used herein interchangeably and refer to a polynucleotide such as DNA having at least 70% sequence identity to the parent polynucleotide. The variant may include mutations such as deletion, addition or substitution such that the mutations do not change the open reading frame and the polynucleotide encodes a peptide or a protein having substantially similar structure and function as a peptide or a protein encoded by the parent polynucleotide. According to some embodiments, the variants are conservative variants. The term “conservative variants” as used herein refers to variants in which a change of one or more nucleotides in a given codon position results in no alteration in the amino acid encoded at that position. Thus, the peptide or the protein encoded by the conservative variants has 100% sequence identity to the peptide or the protein encoded by the parent polynucleotide. According to some embodiments, the variant is a non-conservative variant encoding to a peptide or a protein being a conservative analog of the peptide of the protein encoded by the parent polynucleotide. According to some embodiments, the variant has at least 75%, at least 80% at least 85%, at least 90%, at least 95%, at least 98% or at least 99% sequence identity to the original nucleic acid sequence. According to one embodiment, the variant is a conservative variant.

The term “nucleic acid molecule” refers to a single stranded or double stranded sequence (polymer) of deoxyribonucleotides or ribonucleotides. The terms “nucleic acid” and “polynucleotide” are used herein interchangeably. In addition, the polynucleotide includes analogues of natural polynucleotides, unless specifically mentioned. According to an embodiment, the nucleic acid may be selected from the group consisting of deoxyribonucleic acid (DNA), ribonucleic acid (RNA), peptide nucleic acid (PNA), locked nucleic acid (LNA), and analogues thereof, but is not limited thereto. The term encompasses DNA, RNA, single stranded or double stranded and chemical modifications thereof. According to one embodiment, the nucleic acid molecule is DNA.

According to some embodiments, the nucleic acid molecule is an isolated nucleic acid molecule. The term “isolated nucleic acid” as used herein denotes that the nucleic acid is essentially free of other cellular components with which it is associated in the cell. It can be, for example, a homogeneous state and may be dry or in the state of a solution, such as aqueous solution. Purity and homogeneity are typically determined using analytical chemistry techniques such as polyacrylamide gel electrophoresis or high performance liquid chromatography. The term “encoding” refers to the ability of a nucleotide sequence to code for one or more amino acids. The term does not require a start or stop codon. An amino acid sequence can be encoded in any one of six different reading frames provided by a polynucleotide sequence and its complement.

According to some embodiments, the nucleic acid molecule encodes amino acid sequence SEQ ID NO: 15. According to another embodiment, the nucleic acid comprises nucleic acid sequence SEQ ID NO: 21 or a variant thereof having at least 95% sequence identity to the sequence. According to some embodiments, the nucleic acid molecule encodes amino acid sequence SEQ ID NO: 37. According to yet another embodiments, the nucleic acid encodes an analog of amino acid sequence selected from SEQ ID NO: 15 and 37, having at least 95% sequence identity to said sequence.

According to one embodiment, the nucleic acid molecule further encodes amino acid sequence selected from SEQ ID NO: 17, 18, 19, an analog thereof and any combination thereof.

According to some embodiments, the nucleic acid molecule comprises a nucleic acid sequence selected from SEQ ID NO: 22, 23, 24, a variant thereof having at least 95% sequence identity to the original sequence(s), and a combination thereof. According to one embodiment, the nucleic acid sequence comprises nucleic acid sequence SEQ ID NO: 22, 23, and 24. According to one embodiment, the nucleic acid encodes amino acid sequence SEQ ID NO: 20 or an analog thereof having at least 90% sequence identity. According to some embodiments, the nucleic acid molecule of the present invention comprises nucleic acid sequence SEQ ID NO: 25 or a variant thereof having at least 95% sequence identity to the original sequence. According to one embodiment, the nucleic acid encodes amino acid sequence SEQ ID NO: 28 or an analog thereof having at least 90% sequence identity. According to some embodiments, the nucleic acid molecule of the present invention comprises nucleic acid sequence SEQ ID NO: 29 or a variant thereof having at least 95% sequence identity to the original sequence. According to one embodiment, the nucleic acid encodes amino acid sequence SEQ ID NO: 37 or an analog thereof having at least 90% sequence identity. According to another one embodiment, the nucleic acid encodes amino acid sequence SEQ ID NO: 38 or an analog thereof.

According to another aspect, the present invention provides a nucleic acid construct comprising the nucleic acid molecule of the present invention, operably linked to a promoter. According to one embodiment, the nucleic acid construct comprises a nucleic acid molecule comprising nucleic acid sequence SEQ ID NOs: 25 or a variant thereof having at least 95% sequence identity to the original sequence(s) operably bound to a promoter. According to another embodiment, the nucleic acid construct comprises a nucleic acid molecule comprising nucleic acid sequence SEQ ID NOs: 29 or a variant thereof having at least 95% sequence identity to the original sequence(s) operably bound to a promoter.

The term “nucleic acid construct” as used herein refers to an artificially constructed segment of a nucleic acid molecule. It can be an isolate or integrated into another DNA molecule. Accordingly, a “recombinant nucleic acid construct” is produced by laboratory methods.

The terms “operably linked”, “operatively linked”, “operably encodes”, “operably bound” and “operably associated” are used herein interchangeably and refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which then in turn is translated into the protein encoded by the coding sequence.

The term “promoter” as used herein refers to a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to xylose availability. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

According to another aspect, the present invention provides a vector comprising the nucleic acid molecule or nucleic acid construct of the present invention. The terms “vector” and “expression vector” are used herein interchangeably and refer to any viral or non-viral vector such as plasmid, virus, retrovirus, bacteriophage, cosmid, artificial chromosome (bacterial or yeast), phage, binary vector in double or single stranded linear or circular form, or nucleic acid, the sequence which is able to transform host cells and optionally capable of replicating in a host cell. The vector may be integrated into the cellular genome or may exist extrachromosomally (e.g., autonomous replicating plasmid with an origin of replication). The vector may contain an optional marker suitable for use in the identification of transformed cells, e.g., tetracycline resistance or ampicillin resistance. A cloning vector may or may not possess the features necessary for it to operate as an expression vector. Any vector known in the art is envisioned for use in the practice of this invention. According to other embodiments, the vector is a virus, e.g. a modified or engineered virus. The modification of a vector may include mutations, such as deletion or insertion mutation, gene deletion or gene inclusion. In particular, a mutation may be done in one or more regions of the viral genome. Such mutations may be introduced in a region related to internal structural proteins, replication, or reverse transcription function. Other examples of vector modification are deletion of certain genes constituting the native infectious vector such as genes related to the virus' pathogenicity and/or to its ability to replicate. Any virus can be attenuated by the methods disclosed herein. According to some embodiments, the vector is a virus selected from lentivirus, adenovirus, modified adenovirus and retrovirus. In one particular embodiment, the vector is lentivirus.

According to another aspect, the present invention provides a cell comprising the CAR, the nucleic acid molecule, the nucleic acid construct and/or the vector of the present invention. All aspects and embodiments defined above apply herein as well. According to some embodiment, the cell is selected from a bacterial, fungi (such as yeast) and mammalian cell. According to some embodiments, the cell is a mammalian cell. According to another embodiment, the cell is a human cell. According to some embodiment, the cell is lymphocyte. According to some embodiments, the cell is selected from T cell and a natural killer (NK) cell.

The term “T cell” refers to a type of white blood cell that can be distinguished from other white blood cells by the presence of a T cell receptor on the cell surface. There are several subsets of T cells, including, but not limited to, T helper cells (a.k.a. Tx cells or CD4⁺ T cells) and subtypes, including T_{H}1, T_{H}2, T_{H}3, T_{H}17, T_{H}9, and T_{FH} cells, cytotoxic T cells (i.e., Tc cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺FOXP3⁺ Trcells, CD4⁺FOXP3⁻ Trcells, Tr1 cells, Th3 cells, and Tn17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention. According to some embodiments, the cells are T cells. According to some embodiments, the T-cells are selected from memory, regulatory, helper or natural killer T-cells. According to some embodiments, the T cell is selected are from CD4+ T-cell and a CD8+ T-cell. According to some embodiments, the T cell are CD4+ T-cell and a CD8+ T-cell. According to some embodiments, the cells are NK cells. According to some embodiments, the cells are NK T-cells.

According to some embodiments, the present invention provides T-cell comprising the CAR of the present invention having amino acid sequence selected from SEQ ID NO: 20, 28 and 38. According to some embodiments, the cell expresses or capable of expressing the CAR of the present invention. Thus, according to some embodiments, the present invention provides a T-cell genetically modified to express the CAR of the present invention. According to one embodiment, the present invention provides a T-cell genetically modified to express or expressing a CAR comprising amino acid sequence SEQ ID NO: 20. According to another embodiment, the present invention provides a T-cell genetically modified to express or expressing a CAR comprising amino acid sequence SEQ ID NO: 28. According to yet another embodiment, the present invention provides a T-cell genetically modified to express or expressing a CAR comprising amino acid sequence SEQ ID NO: 38.

According to some embodiments, the cell, such as T-cell comprises the nucleic acid molecule encoding the CAR of the present invention. According to other embodiments, the cell, such as T-cell comprises the nucleic acid construct comprising nucleic acid molecule encoding the CAR of the present invention. According to a further embodiment, the present invention provides a vector comprising the nucleic acid construct or molecule encoding the CAR of the present invention. According to such embodiments, the T-cell is capable of expressing or expresses the CAR of the present invention.

According to another aspect, the present invention provides a composition comprising a plurality of cells of the present invention and a carrier. According to some embodiments, the present invention provides a composition comprising a plurality of CARs of the present invention. All aspects and embodiments defined above apply herein as well. According to some embodiments, the composition is a pharmaceutical composition. According to some embodiments, the present invention provides a pharmaceutical composition comprising a plurality of CARs of the present invention and a pharmaceutically acceptable carrier. According to other embodiments, the present invention provides a pharmaceutical composition comprising a plurality of cells of the present invention and a pharmaceutically acceptable carrier. The plurality of cells may also be referred to as a cell composition. According to some embodiments, the pharmaceutical composition comprises a plurality of T-cells expressing the CAR of the present invention having amino acid sequence SEQ ID NO: 20. According to other embodiments, the pharmaceutical composition comprises a plurality of T-cells expressing the CAR of the present invention having amino acid sequence SEQ ID NO: 28. According to some embodiments, the pharmaceutical composition comprises a plurality of T-cells expressing the CAR of the present invention having amino acid sequence SEQ ID NO: 38. According to some embodiments, the pharmaceutical composition comprises a plurality of T-cells capable of expressing the CAR of the present invention having amino acid sequence selected from SEQ ID NO: 20, 38 and 38. According to another embodiment, the present invention provides the pharmaceutical compositions comprising a plurality of T-cells comprising the nucleic acid molecule, construct or vector and capable of expressing the CAR of the present invention. According to some embodiments, the T-cells are CD8+ T-cells. According to other embodiments, the T-cells are CD4+ T-cells. According to some embodiments, the T-cells are a combination of CD4+ and CD8+ cells.

The term “pharmaceutical composition” as used herein refers to a composition comprising at least one active agent as disclosed herein, e.g. CAR or CAR T-cells, formulated together with one or more pharmaceutically acceptable carriers.

Formulations of the pharmaceutical composition may be adjusted according to applications. In particular, the pharmaceutical composition may be formulated using a method known in the art so as to provide a rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The pharmaceutical compositions of the present invention may be prepared by conventional techniques, e.g., as described in Remington: The Science and Practice of Pharmacy, 19th Ed., 1995. The compositions may be in solid, semisolid or liquid form and may further include pharmaceutically acceptable fillers, carriers or diluents, and other inert ingredients and excipients. The compositions can be administered by any suitable route, e.g., orally, intravenously, parenterally, rectally or transdermally, the oral route being preferred. The dosage will depend on the state of the patient and will be determined as deemed appropriate by the practitioner.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. solid carriers or excipients such as, for example, lactose, starch or talcum or liquid carriers such as, for example, water, fatty oils or liquid paraffins.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerine and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

According to some embodiments, the composition is formulated for a parenteral administration. According to one embodiment, the composition is formulated for subcutaneous, intraperitoneal (IP), IM, IV and intratumor administration. According to other embodiments, the composition is formulated as a solution such as a sterile solution for injection.

According to any one of the above embodiments, the pharmaceutical composition of the present invention is for use in treating cancer. According to some embodiments, the use comprises administering the pharmaceutical composition to a subject. According to some embodiments, the cancer is cancer overexpressing SLeA glycan. According to one embodiment, the cancer is selected from hematological, breast, ovarian, pancreatic, colorectal, stomach, liver, lung, oropharyngeal cancer, squamous cell carcinoma, head and neck and gallbladder cancer, and any other SLeA-positive (SLeA-presenting or SLeA-expressing) cancers. According to some embodiment, the cancer is a breast cancer. According to some embodiment, the cancer is a Her-2 negative breast carcinoma. According to another embodiment, the cancer is an ovarian cancer. According to a further embodiment, the cancer is a colon cancer. According to one embodiment, the cancer is colon adenocarcinoma. According to another embodiment, the cancer is a stomach cancer. According to one embodiment, the cancer is a pancreatic cancer. According to another embodiment, the cancer is a pancreatic adenocarcinoma. According to yet another embodiment, the cancer is lung cancer. According to one embodiment, the cancer is lung adenocarcinoma. According to some embodiments, the cancer is squamous cell carcinoma. According to another embodiment, the cancer is pharynx squamous cell carcinoma. According to one embodiment, the cancer is a hematological cancer overexpressing SLeA glycan.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

The term “treating cancer” as used herein should be understood to e.g. encompass treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer, inhibition of growth and proliferation of cancer cells and so forth. The term “treating cancer” as used herein should also be understood to encompass prophylaxis such as prevention as cancer reoccurs after previous treatment (including surgical removal) and prevention of cancer in an individual prone (genetically, due to life style, chronic inflammation and so forth) to develop cancer. As used herein, “prevention of cancer” is thus to be understood to include prevention of metastases, for example after surgical procedures or after chemotherapy.

According to some embodiments, the use comprises administering the pharmaceutical composition to a subject. According to any one of the above embodiments, the composition of the present invention is administered as known in the art. According to one embodiment, the composition is parenterally administered, e.g. IP, IV, IM, SC or intratumorally. According to some embodiments, the pharmaceutical composition is administered via infusion.

The terms “administering” or “administration of” a substance, a compound or an agent to a subject are used herein interchangeably and refer to a an administration mode can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the pharmaceutical composition is parenterally administered. The term “parenteral” refers to subcutaneous, intracutaneous, intravenous, intramuscular, intraarticular, intraarterial, intrasynovial, intrasternal, intrathecal, intralesional, intraperitoneal and intracranial injection, as well as various infusion techniques.

According to some embodiments, the pharmaceutical composition of the present invention comprising the CAR T-cells of the present invention is co-administered with an additional anti-tumor therapy including but not limited to anticancer drugs, radiotherapy, immunotherapy and surgery. According to some embodiments, the pharmaceutical composition is co-administered with another anti-cancer drug. According to some embodiments, the therapeutic agents suitable in an anti-neoplastic composition for treating cancer include, but not limited to, chemotherapeutic agents, radioactive isotopes, toxins, cytokines such as interferons, immunostimulating agents, immunomodulating agents and antagonistic agents targeting cytokines, cytokine receptors or antigens associated with tumor cells. In some embodiments, the anti-cancer agent is a chemotherapeutic.

The term “co-administration” encompasses the administration of a first and second agent in an essentially simultaneous manner, such as in a single dosage form, e.g., a capsule or tablet having a fixed ratio of first and second amounts, or in multiple dosage forms for each. The agents can be administered in a sequential manner in either order. When co-administration involves the separate administration of each agent, the agents are administered sufficiently close in time to have the desired effect (e.g., complex formation). The term “anti-cancer”, “anti-neoplastic” and “anti-tumor” when referred to a compound, an agent or a moiety are used herein interchangeably and refer to a compound, drug, antagonist, inhibitor, or modulator such as immunomodulatory having anticancer properties or the ability to inhibit or prevent the growth, function or proliferation of and/or causing destruction of cells,” and in particular tumor cells. Therapeutic agents suitable in an anti-neoplastic composition for treating cancer include, but not limited to, chemotherapeutic agents, radioactive isotopes, toxins, cytokines such as interferons, immunostimulating agents, immunomodulating agents and antagonistic agents targeting cytokines, cytokine receptors or antigens associated with tumor cells. In some embodiments, an anti-cancer agent is a chemotherapeutic.

The T-cells of the present invention are capable of expressing the CAR molecules encoded by the DNA or RNA by which the T-cells are transduced infected or electroporated.

According to another aspect, the present invention provides a method for treating cancer in a subject in need thereof comprising administering to the subject a therapeutically effective amount of cells or the pharmaceutical composition of the present invention. According to some embodiments, the cells are engineered cells expressing the CAR of the present invention. All aspects and embodiments defined above apply herein as well. According to other embodiments, the cells are engineered to express the CAR of the present invention. According to some embodiments, the cells are CAR T-cells. According to some embodiments, the cells are NK cells comprising CAR the CAR of the present invention. The term “therapeutically effective amount” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation. According to some embodiments, the

According to yet another aspect, the present invention provides use of cells, such as T cells, comprising the CAR of the present invention in the preparation of a medicament for treating cancer. According to other embodiments, the present invention provides use of the CARs of the present invention in the preparation of a medicament for treating cancer.

The term “consisting essentially of” means that the composition or component may include additional ingredients, but only if the additional ingredients do not materially alter the basic and novel characteristics of the claimed compositions or methods.

Having now generally described the invention, the same will be more readily understood through reference to the following examples, which are provided by way of illustration and are not intended to be limiting of the present invention.

### EXAMPLES

Methods

Antibody Purification

Human embryonic kidney 293A cells were used to produce whole Koprowski's (Native) and RA9-23 antibodies using polyethylenimine (PEI; Polysciences) reagent as described before. Antibodies were purified using protein A (GE healthcare) and concentrations were determined by BCA assays (Pierce). For higher antibody amounts, p3BNC plasmids with variable region of Ab RA9 upstream to human IgG1 heavy and light constant regions were transfected into HEK293F cells using PEI max as a transfection reagent (Polysciences). Medium sup was collected six days post transfection, centrifuged and filtered with the addition of PMSF and azide. Medium was loaded on a protein-A column (GE lifesciences), eluted with 0.1M citric acid pH3 and brought to pH7 with 2M tris buffer pH8.

Antibodies K_{D}

Polyvalent binding studies were carried out using the Octet Red system (ForteBio, Version 8.1, Menlo Park, Calif., USA, 2015) that measures biolayer interferometry (BLI). All steps were performed at 30° C. with shaking at 1500 rpm in a black 96-well plate containing 200 μL solution in each well. Streptavidin-coated biosensors were loaded with 50 nM of biotinylated SLe^{a}-PAA (or biotinylated Le^{a}-PAA, as a negative control) for 300 s followed by washing step [with PBS buffer, pH 7.4, containing 1 mg/ml BSA and 0.1% (v/v) Tween 20]. Sensors were then reacted for 300 s with each antibody (native and selected clones) at increasing concentrations from 25 to 100 nM and then moved to buffer-containing wells for another 300 s (dissociation phase). Binding and dissociation were measured as changes over time in light interference after subtraction of parallel measurements from unloaded biosensors. Sensorgrams were fitted with a 1:1 binding model using the Octet data analysis software 8.1 (Fortebio, Menlo Park, Calif., USA, 2015).

ELISA

Binding of antibodies to various glycans was tested by ELISA. Glycans (Glycotech) were coated in duplicates at 0.25 μg/well in 50 mM sodium carbonate-bicarbonate buffer, 25 pH 9.5 onto 96-well microtiter plates (Costar, Corning) and plates were incubated overnight at 4° C. Wells were blocked for 1 hour at room temperature with blocking buffer [PBS pH 7.4, 1% ovalbumin (Grade V, Sigma)]. Blocking buffer was removed and primary antibody was added at 10 μg/ml in 100 μl/well in the same blocking buffer for two hours at room temperature. The plates were washed three times with PBST (PBS pH 7.4, 0.1% Tween) and subsequently incubated for 1 hour at room temperature with HRP-goat anti-human IgG 0.11 μg/ml in PBS. After washing three times with PBST, wells were developed with 140 μl of O-phenylenediamine in 100 mM citrate-PO₄ buffer, pH 5.5, and the reaction stopped with 40 μl of H₂SO₄ (4 M). Absorbance was measured at 490 nm on SpectraMax M3 (Molecular Devices). Specific binding was defined by subtracting the background readings obtained with the secondary antibody only on wells coated with PAA. For ELISA inhibition assay, 96 well plate was coated with SLeA-PAA-Biotin (GlycoTech) in triplicates at 0.25 μg/well overnight at 4° C. Wells were blocked with blocking buffer. The RA9-23 antibody at 0.16 μg/mL was pre-incubated with either specific or non-specific target antigens (SLeA-PAA-Biotin and LeA-PAA-Biotin or SLeX-PAA-Biotin glycans, respectively) at 300-0.3 nM in blocking buffer. Antibody-glycan mixtures were incubated at 4° C. for two hours. Blocking buffer was removed from plate and antibody-glycan mixtures were added to the respective wells at 100 μL/well in triplicates, then incubated for two hours at room temperature, followed by washing, secondary antibody and substrate developing, as described above.

Cell Culture

WiDr and Capan2 cells (human colorectal and pancreatic cancer cell lines, respectively) were obtained from American Type Culture collection (ATCC). WiDr and Capan2 cells were grown in DMEM (biological industries) supplemented with 10% heat inactivated fetal bovine serum (FBS), 2 mM L-glutamine, 100 units/ml penicillin and 0.1 mg/ml streptomycin.

Cancer Cells Binding Assay

WiDr and Capan2 cells (human colorectal and pancreatic cancer cell lines, respectively) were collected from plates using 10 mM EDTA. Cells were incubated with native and RA9-23 antibodies diluted in PBS+0.5% fish gelatin for 1 hour on ice, followed by incubation with Cy3 AffiniPure Goat Anti-Human IgG (H+L) (Jackson) diluted 1:100 in PBS+0.5% fish gelatin for 1 hour on ice. Fluorescence of cells was measured by CytoFLEX flow cytometry (Beckman Coulter).

For sialidase FACS assay, WiDr cells were collected from plates using 10 mM EDTA. 0.5×10⁶ cells were divided into Eppendorf tubes and incubated for four hours at 37° C. with either PBS, 50 mU active Arthrobacter Ureafaciens Sialidase (AUS) (EY Laboratories, San Mateo, Calif., USA) or 50 mU inactive AUS (pre-incubated in 90° C. for 30 min) in PBS. Then, cells were washed with FACS buffer, stained with 2.5 μg/mL RA9-23 antibody, followed by washing, secondary antibody labeling and fluorescence measurement, as described above.

Cell Lines and Culture Cell Lines

293T human embryonic cells (ATCC; CRL-1573), FaDu pharynx squamous cell carcinoma cells (ATCC; HTB43), and packaging cell lines and PG13 (ATCC; CRL-10686) were cultured in DMEM supplemented with 10% fetal calf serum (FCS), 2 mM glutamine and 1 mM sodium pyruvate. Human lymphocytes were cultured in RPMI-1640 (Biological Industries) supplemented with 10% FCS, 2 mM glutamine. All media were supplemented with a mixed antibiotic solution containing penicillin (100 U/ml), streptomycin (100 μg/ml) and neomycin (10 μg/ml) (Bio-Lab). Primary human cells were from Cell Biologics and Lonza. Each primary cell was propagated according to the manufacturer's instructions. The cells were incubated in a humidified 37° C. incubator with 5% CO₂, except for the PG13, which were kept with 7.5% CO₂. All cells were verified to lack mycoplasma by PCR (HyLabs). The cells were frozen at low passage, and the number of passages after thawing was recorded. Cells were maintained in culture for no longer than 4 weeks, which corresponds to approximately 12 passages.

CDC Assay

For complement-dependent cytotoxicity (CDC) we used rabbit complement (Sigma). Cytotoxicity was evaluated by measuring lactate dehydrogenase (LDH) release using LDH Cytotoxicity Detection kit (Roche Applied Science) according to the manufacturer's instructions. All assays included maximum release control contains rabbit complement diluted 1:6 with 1% TritonX-100. For spontaneous release control, cells were incubated only with rabbit complement. Percentage cytotoxicity was calculated as: (test release-spontaneous release)/(maximum release-spontaneous release)×100. 2×10⁴ target Cells were incubated in triplicates with antibodies at 20 and 2 ng/μl for 1 hour on ice in 96-well round-bottom plates. Rabbit complement and triton were added and plates were incubated for 2 hours at 37° C. Then supernatants were collected and LDH release was determined.

Sialoglycan Microarray Fabrication

Arrays were fabricated with NanoPrint LM-60 Microarray Printer (Arrayit) on epoxide-derivatized slides (Corning 40044) with 16 sub-array blocks on each slide. Glycoconjugates were distributed into one 384-well source plates using 4 replicate wells per sample and 8 μl per well (Version 2.0). Each glycoconjugate was prepared at 100 μM in an optimized print buffer (300 mM phosphate buffer, pH 8.4). To monitor printing quality, replicate-wells of human IgG (80, 40, 20, 10, 5, 0.25 ng/μl in PBS+10% glycerol) and AlexaFlour-555-Hydraside (Invitrogen A20501MP, at 1 ng/μl in 178 mM phosphate buffer, pH 5.5) were used for each printing run. The arrays were printed with four 946MP3 pins (5 μm tip, 0.25 μl sample channel, ˜100 μm spot diameter; Arrayit). Each block (sub-array) has 20 spots/row, 20 columns with spot to spot spacing of 275 μm. The humidity level in the arraying chamber was maintained at about 70% during printing. Printed slides were left on arrayer deck over-night, allowing humidity to drop to ambient levels (40-45%). Next, slides were packed, vacuum-sealed and stored at room temperature (RT) until used.

Sialoglycan Microarray Binding Assay

Slides were developed and analyzed as previously described by Padler-Karavani (J Biol Chem. 2012; 287: 22593-22608) with some modifications. Slides were rehydrated with dH₂O and incubated for 30 min in a staining dish with 50° C. pre-warmed ethanolamine (0.05 M) in Tris-HCl (0.1 M, pH 9.0) to block the remaining reactive epoxy groups on the slide surface, then washed with 50° C. pre-warmed dH₂O. Slides were centrifuged at 200×g for 5 min then fitted with ProPlate™ Multi-Array 16-well slide module (Invitrogen) to divide into the sub-arrays (blocks). Slides were washed with PBST (0.1% Tween 20), aspirated and blocked with 200 μl/sub-array of blocking buffer (PBS/OVA, 1% w/v ovalbumin, in PBS, pH 7.3) for 1 hour at RT with gentle shaking. Next, the blocking solution was aspirated and 100 μl/block of purified antibodies in 20-1.28×10⁻⁴ ng/μl diluted in PBS/OVA were incubated with gentle shaking for 2 hours at RT. Slides were washed three times with PBST, then with PBS for 2 min. Bound antibodies were detected by incubating with secondary detection diluted in PBS, 200 μl/block at RT for 1 hour, Cy3-anti Human IgG 0.4 μg/ml (Jackson Immunoresearch). Slides were washed three times with PBST then with PBS for 10 min followed by removal from ProPlate™ Multi-Array slide module and immediately dipping in a staining dish with dH₂O for 10 min with shaking, then centrifuged at 200×g for 5 min. Dry slides immediately scanned.

Array Slide Processing and Apparent K_{D} Calculations

Processed slides were scanned and analyzed as described at 10 μm resolution with a Genepix 4000B microarray scanner (Molecular Devices) using 350 gain. Image analysis was carried out with Genepix Pro 6.0 analysis software (Molecular Devices). Spots were defined as circular features with a variable radius as determined by the Genepix scanning software. Local background subtraction was performed. Apparent K_{D} was calculated according to non-linear fit with one-site specific binding using GraphPad Prism 8.0.

Construction of Chimeric Antigen Receptor (CAR) and Retroviral Vector Production

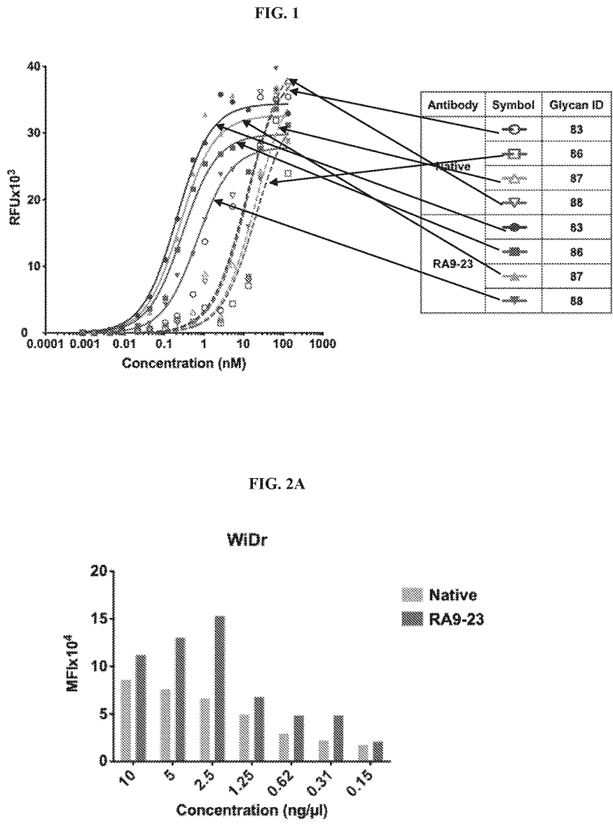

The CAR construct contained a leader signal peptide, RA9-23 scFv (VH connected to the VL through 3×G₄S spacer), strep-tag, 2×G₄S spacer, human CD28 gene (hCD28 cytoplasmic, transmembrane and co-stimulation domains) followed by the human FcγR ITAM signaling domain (FIG. 1; Seq in the attached file). The sequence was cloned into the retroviral vector pMSGV1 (Hughes et al., 2005).

In order to produce a stable packaging cell line, 293T cells were co-transfected with retroviral vector plasmids (Gag-Pol) and the plasmid of interest (pMSGV1-CAR T) using CaPO₄, as described (Elinav et al., 2009). Supernatants containing the retrovirus were collected 16 hours later and used to stably transduce the amphotropic PG13 packaging cells. Cells were sorted by FACSort flow Cytometer (BD PharMingen) to achieve 100% RA9-CAR⁺-PG13 expressing cells, re-grown and frozen at −80° C.

T Cell Transduction

T cell transduction was done as previously described (Maliar et al., 2012). Briefly, peripheral human blood lymphocytes (PBL) were isolated from the blood of healthy human donors by density gradient centrifugation on Ficoll-Paque (Axis-shield). PBLs were activated in non-tissue culture-treated 6-well plates pre-coated with both mouse-anti-human CD3 (prepared in-house from hybridoma OKT3) and mouse-anti-human CD28 for 48 hours at 37° C. Activated lymphocytes were harvested, divided into two groups then co-cultured for 48 hours with 100 IU/mI IL-2 (untransduced cells) or for two consecutive retroviral transductions in RetroNectin (Takara Shuzu Ltd.) that was pre-coated to non-tissue culture-treated 6-well plates supplemented with 100 IU/ml human IL-2 (Novartis Pharma GMbH). At the end of transduction, both untransduced and transduced T cells were cultured in the presence of 350 IU/ml IL-2 for 24-72 hours for in-vilro or in-vivo assays, respectively. Transduction efficiency was monitored by flow cytometry analysis using FITC-mouse-anti-strep-tag IgG1 according to manufacturer instructions.

Cytokine Production

A total of 1×10⁶ untransduced or RA9-23 CAR transduced T cells were co-cultured with 0.5-10⁶ of cells (FaDu, OVCAR8 or primary human cells) in 24-wells for 16 hours in a RPMI medium supplemented with 10% FCS, 2 mM glutamine and antibiotics. The cell-free growth medium was collected and analyzed for IFN-γ production by ELISA using a human IFN-γ ELISA kit, according to the manufacturer's instructions (R&D systems).

Viability Assay/Methylene Blue Dye Staining

A total of 0.5×10⁶ of FaDu cells were co-cultured in a 24-well plate with 1-, 5-, 10-fold amounts of T cells (untransduced or RA9-23 CAR T cells) for 16 hours. The plate was washed with PBS to remove T cells and FaDu dead cells. The remaining cells were fixed with 4% formaldehyde (2 h, RT) and stained with 0.5% methylene blue (Sigma Aldrich) for 15 min at RT. The plate was washed with ddH₂O and 0.1 M HCL was added prior to analysis. The 620 nm absorbance was read on a Multiskan FC ELISA reader (Thermo Fisher Scientific). The viability of FaDu cells, post co-culturing with T cells, was evaluated based on the Methylene Blue Dye reduction and in comparison to the value of FaDu cells cultured with no lymphocytes that served as 100% viability (% viability=[value of FaDu cells co-cultured with lymphocytes]/[value of FaDu cells cultured without lymphocytes]×100%).

Flow Cytometry

Cells were suspended in FACS buffer (5% FCS, 0.05% sodium azide in PBS) and incubated with antibodies for 30 min at 4° C. in the dark, washed prior to either analysis or staining with corresponding secondary antibodies (30 min, 4° C.). Samples were collected with FACScanto (BD PharMingen) and analyzed with FCS express Software (De Novo Software). 50,000 total events were recorded per sample.

CAR T Glycan Specificity

RA9-23 CAR T Cells and N29 CAR T Cells (served as irrelevant control CAR T cells) were incubated with 1 μM biotinylated-polyacrylamide conjugated glycans (Glycotech; 6-8 glycans per Bio-PAA molecule) diluted in PBS+0.5% fish gelatin (FACS buffer) for 45 minutes on ice, followed by incubation with APC-Streptavidin (Southern Biotech) diluted 1:1000 in FACS buffer for 30 minutes on ice. Cells were washed in FACS buffer and cell fluorescence was measured by CytoFLEX flow cytometry (Beckman Coulter).

Adoptive Cell Transfer

NSG (NOD.Cg-Prkdcscid Il2rgtmlWjl/SzJ) were obtained from Jackson Laboratories (Maine, USA) and maintained in a Specific Pathogen-Free Facility of the Tel Aviv Sourasky Medical Center. The head and neck squamous cell carcinoma (HNSCC) model was generated by subcutaneous injection of 200 μl 0.5×10⁶ FaDu cells (resuspended in a mixture of PBS and growth factor-reduced Matrigel; BD Biosciences; at 1:1 ratio) into the flanks of 8 to 10-week-old females mice. On day 11, mice were randomly assigned into experimental groups and irradiated at 2Gy. On the following day, FaDu tumor-bearing mice were adoptively transferred with untransduced or RA9-23 CAR T cells via intravenous (10⁷ T cells in 500 μl PBS) or intra-tumoral (107 T cells in 200 μl PBS) injections. Tumor growth was monitored by a caliper twice a week through ˜40 days, and tumor volume calculated (tumor volume mm³=[length mm×(width mm)²]/2; n=5 per group).

Statistical Analysis

Data was analyzed and graphed using Graphpad Prism V.6 (San Diego, Calif., USA),as indicted in context. P value of 0.05 was considered statistically significant.

### Example 1. Affinity Assessment of Whole RA9-23 Antibody

Whole RA9-23 antibodies were produced by cloning the VH and VL domains into p3BNC human IgG1 expression vectors. The sequences of the VH and HL chains are provided below.

Heavy chain:
KVKLEESGGG LVQPGGSMKL SCAASGFTFS DAWMDWVRQS
PEKGLEWVAE IGNKGNNHAT NYAESVKGRF TVSRDDSKSR
VYLQMNSLRV EDTGTYYCTT RFAYW GQGTP VTVPA
Light chain:
DIKMTQSPSS MYASLGERVT IPCKASQDIN SYLSWFQQKP
GKSPKTLIYR ANRLVDGVPS RFSGSGSGQD YSLTISSLEY
EDMGIYYCLQ YDEFPRTFGG GTKLEIK

It is well known that CDRs may be defined in different methods. According to Kabat the CDR 1, 2, and 3 of the heavy chain have amino acid sequences: DAWMD; EIGNKGNNHATNYAESVKG and RFAY, respectively, and the CDRs 1, 2, and 3 of the light chain have amino acid sequences KASQDINSYLS; RANRLVD; and LQYDEFPRTF, respectively.

Full antibodies were produced in 293A cells and purified with protein A. The antibody binds specifically to SLeA. K_{D} of the whole antibody was also determined by antibody binding kinetics (surface plasmon resonance with Biacore), using polyvalent biotinylated SLea-PAA as antigen. The whole antibody has K_{D} of 1.2×10⁻⁸ M, which is 3.5 lower than the K_{D} of the antibody of Koprowski.

### Example 2. Full-Length Antibodies Specificity

The whole RA9-23 antibody showed that sialic acid is imperative for antigen binding, since the binding to the Lea antigen that lacks the sialic acid was very low (less than 3.3%). Additionally, even though SLe^{a} and SLex contain the same four building blocks, they are perceived as completely different structure, and there is very low cross reactivity (less than 1.8%). This is a striking example of how glycan-linkages are important and critical in glycan diversity and complexity in nature. Altogether, these results demonstrate that selected anti-SLe^{a} antibodies are highly specific and of high affinity.

For more detailed specificity analysis we used glycan microarrays to determine the specificity in a high-throughput assay which contains 88 different glycans. The array analysis showed that the antibodies are very specific to AcSLe^{a}, GcSLe^{a} and their corresponding 9-O-acetylated versions. The importance of the sialic acid and fucose residues for the antibody recognition was also demonstrated, as Le^{a} and Neu5Ac-α-2-3-Galβ1-3GlcNAcβProNH2 (non-fucosylated SLe^{a}) were not detected at all.

Further evaluation of affinities of these antibodies by saturation curves on the glycan microarrays showed that RA9-23 antibody had more than 55 fold higher affinity than the antibody of Koprowski against AcSLe^{a} (see FIG. 1 and Table 3). Even greater affinity improvement of >70 fold was measured against GcSLe^{a} (from 19.8±8.8 to 0.28±0.05) and 9-O-AcSLe^{a} (from 19.9±7.7 to 0.25±0.07).

TABLE 3
The affinity of RA9-23 antibodies to SLe^{a} glycans

| Antibody | Glycan | K_{D} (nM) | SD |
|---|---|---|---|
| Koprowski's | Neu5Ac-SLe^{a} | 11.3 | 5.5 |
| Antibody | Neu5Gc-SLe^{a} | 19.8 | 8.8 |
| | 9-O-Neu5Ac-SLe^{a} | 19.9 | 7.7 |
| | 9-O-Neu5Gc-SLe^{a} | 13.64 | 5.9 |
| RA9-23 | Neu5Ac-SLe^{a} | 0.2 | 0.03 |
| | Neu5Gc-SLe^{a} | 0.28 | 0.05 |
| | 9-O-Neu5Ac-SLe^{a} | 0.25 | 0.07 |
| | 9-O-Neu5Gc-SLe^{a} | 0.67 | 0.29 |

All glycans present in Table 3 are tumor-associated carbohydrate antigens. SLe^{a} can be either populated by Neu5Ac or by the non-human sialic acid Neu5Gc. GcSLe^{a} and 9-O-GcSLe^{a} are expected to appear more in cancer.

The specificity of the whole length antibody was further demonstrated by ELISA inhibition assay, in which binding of RA9-23 to SLeA was inhibited only with the specific glycan SLeA, but not with the closely-related glycans SLeX or Lea (FIG. 8).

### Example 3. Cancer Cell Lines Binding and Cytotoxicity

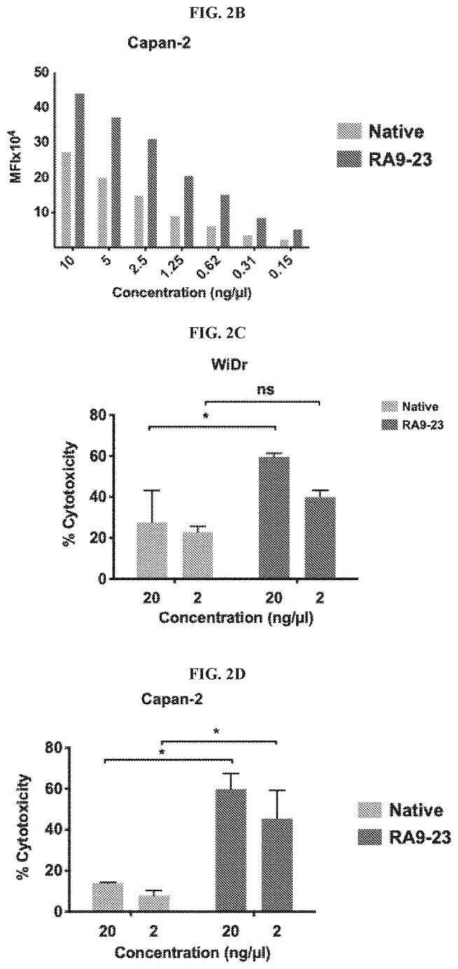

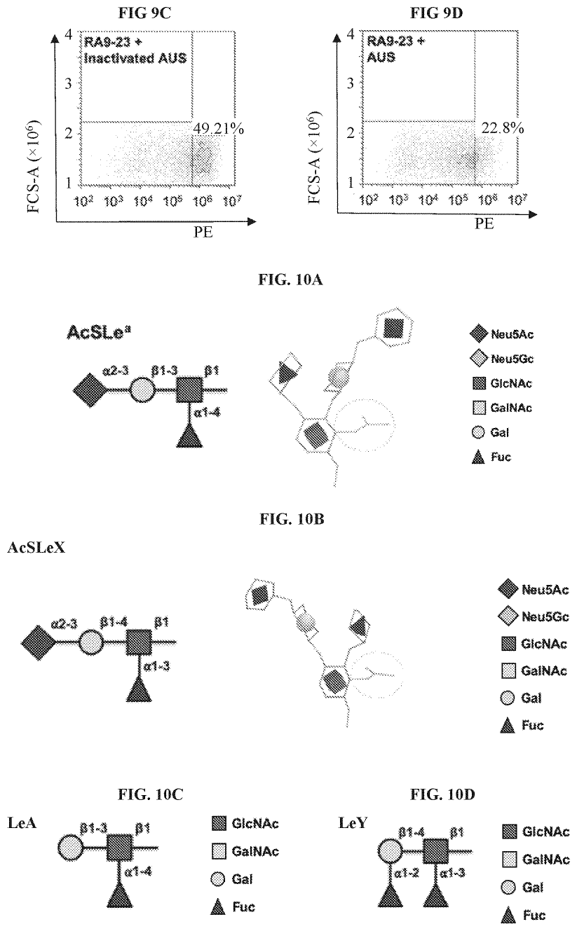

The mutated whole antibody RA9-23 showed improved binding of glycans by various methods, at different glycan densities (e.g. FACS, ELISA, glycan microarrays), and under flow (Biacore). Next, it was examined whether this is reflected in better target recognition in the natural context of cancer cells. Cancer cell binding is critical for antibody therapeutic and diagnostic utilities. We compared the binding of Koprowski's (native) and RA9-23 antibodies to several SLe^{a}-positive cancer cell lines (WiDr and Capan2: human colorectal and pancreatic cancer cell lines, respectively). RA9-23 antibodies showed better binding efficacy than the native antibody in both cell lines and at various concentrations (FIGS. 2A and 2B). These results indicate that RA9-23 antibody has higher affinity not only in mono/polyvalent-glycans settings (FACS, ELISA, glycan microarrays), but also in the context of the whole cell. Typically cells do not uniformly express glyco-conjugates but rather have them heterogeneously distributed over the cell surface. Better cell binding could potentially lead to improved killing of cancer cells. Antibodies of IgG1 isotype are known to be able to facilitate cell killing by complement recruitment (by CDC). The CDC killing potential of the antibodies was evaluated, revealing that the RA9-23 antibodies had a higher cytotoxicity in both WiDr and Capan2 cell lines compared to the native antibody (FIGS. 2C and D). As can be seen in FIG. 9, the binding of the antibody SLeA was reduced dramatically after removal of sialic acids from the cell surface by a sialidase treatment.

### Example 4. RA9-23 CAR T Cells Specificity Against SLeA-Expressing Cancer Cells

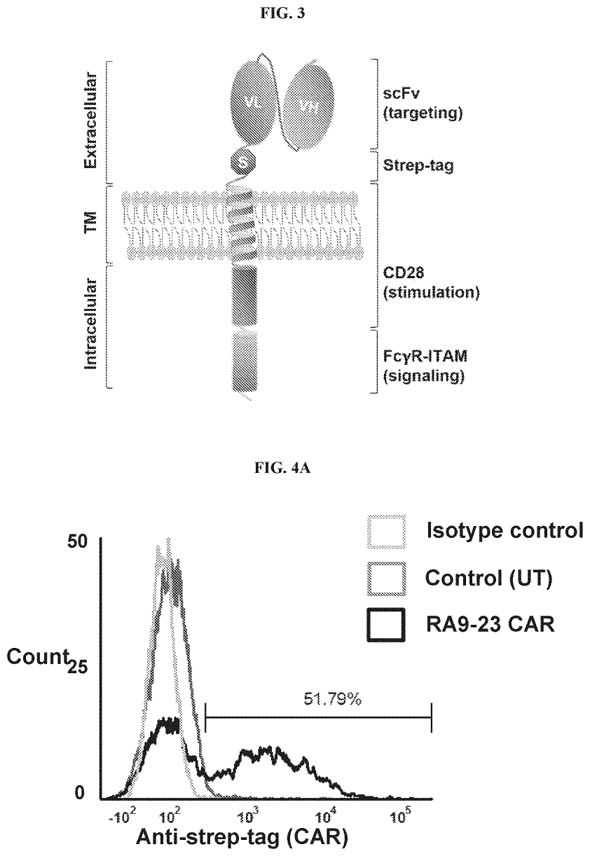

We synthesized single chain variable fragment (scFv) of RA9-23 antibody by linking the variable heavy and variable light chains of the RA9-23 antibody (see the sequences in Example 1) with a 3×(GGGGS) spacer (SEQ ID NO: 33) to obtain amino acid sequence SEQ ID NO: 15. The scFv was incorporated into a CAR backbone containing a strep-tag connected through a 2×(GGGGS) spacer (SEQ ID NO: 32) to the human CD28 transmembrane domain and intracellular co-stimulatory domain (SEQ ID NO: 17), followed by the FcγR ITAM intracellular signaling domain (SEQ ID NO: 18) (schematic representation is shown in FIG. 3). A signaling peptide (SEQ ID NO: 19) is placed at the N-terminus of the construct. The extracellular strep-tag allows to monitor CAR surface expression upon transduction. The CAR construct was cloned into the pMSGV1 retroviral vector, expressed in 293T cells followed by generation of the PG-13 packaging cell line.

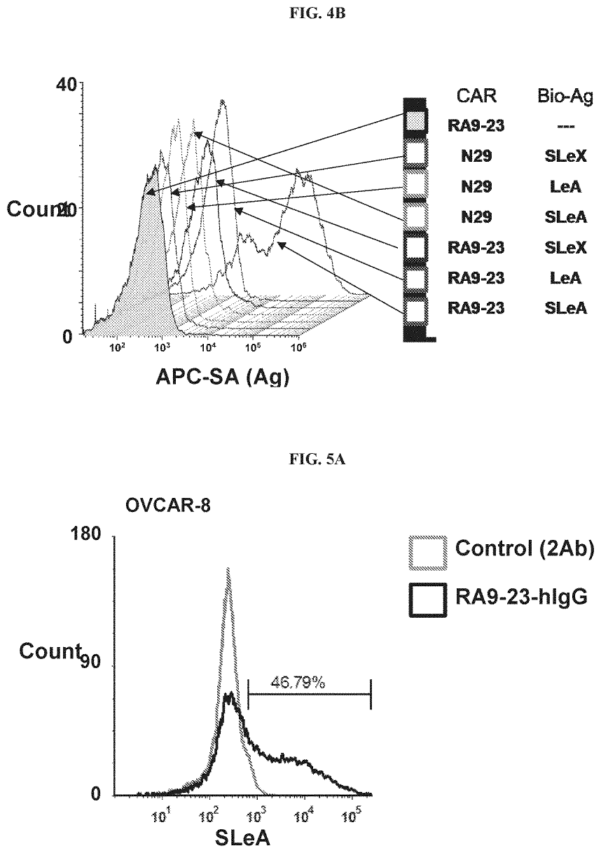

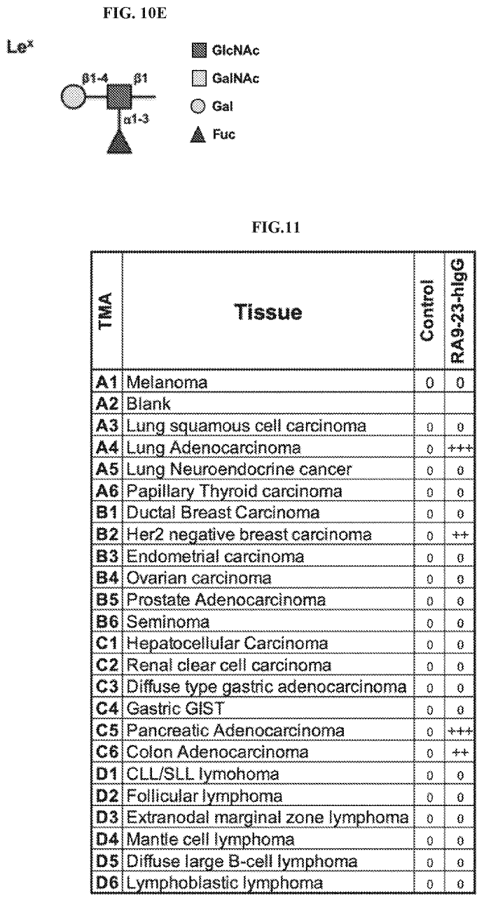

Normal human donor blood lymphocytes were isolated and activated with monoclonal anti-CD3 (clone OKT3) and anti-CD28. Activated T cells were then transduced twice (day after day) with RA9-23-CAR expressing retrovirus, or grown in IL-2-containing media (untransduced; UT), followed by further acclimation in IL-2. FACS analysis against the extracellular strep-tag demonstrated surface expression of CAR only in the transduced cells (RA9-23 CAR), but not in the control UT T cells (FIG. 4A). To evaluate the CAR specificity against SLeA in the context of cell expression, binding of T cells transduced with RA9-23 CAR was examined by FACS against different glycan targets, in comparison to untransduced T cells or control irrelevant-CAR (N29 CAR; targeting ErbB2) transduced T cells (FIG. 4B). The binding was examined against polyvalent-glycans conjugated to biotinylated-polyacrylamide (6-8 glycan units per PAA-Bio) that mimic cancer cell surface expression, using the SLeA tetra-saccharide (Neu5Acα2-3Galβ1-3(Fucα1-4)GcNAcβ1-R), or the closely related antigens: LeA tri-saccharide (Galβ1-3(Fucα1-4)GlcNAcβ1-R) that is missing the terminal sialic acid, and the SLeX (Neu5Acα2-3Galβ1-4(Fucαl-3)GcNAcβ1-R) that differs in the linkages between the sugar units (FIG. 10). While the control UT and irrelevant-N29 CAR T cells did not bind any of the target glycans, the transduced T cells expressing RA9-23 CAR showed binding only to SLeA but not LeA or SLeX (FIG. 4B). Thus, transduction of RA9-23 CAR results in T cells that can specifically target SLeA cancer-associated carbohydrate antigens.

We next characterized the cancer-specificity of RA9-23 CAR transduced T cells on a collection of primary human cells in comparison to control untransduced T cells (FIG. 5). First, we showed that SLeA is expressed on the ovarian carcinoma cell line OVCAR-8 using the RA9-23 monoclonal antibody (FIG. 5A). Thus, OVCAR-8 served as the positive control for cytokine expression in other examples. Primary human cells (alveolar/pancreatic/cardiac endothelial cells/erythrocytes/kidney epithelial cells) and OVCAR-8 cells were co-cultured with RA9-23 CAR T cells or ustransduced T cells, and induction of cytokine secretion to the media was examined. While OVACR-8 cells showed high levels of IFN-γ secretion, minimal cytokine levels were detected after incubation with both the RA9-23 CAR T cells or ustransduced T cells (FIG. 5B). These results support the cancer-specific expression of SLeA and minimal/non-existent expression of this epitope on the cellular surface of the examined normal cells, together suggesting very low risk for off-target cytotoxicity.

### Example 5. RA9-23 CAR T Cells Induce In Vitro Cytotoxicity

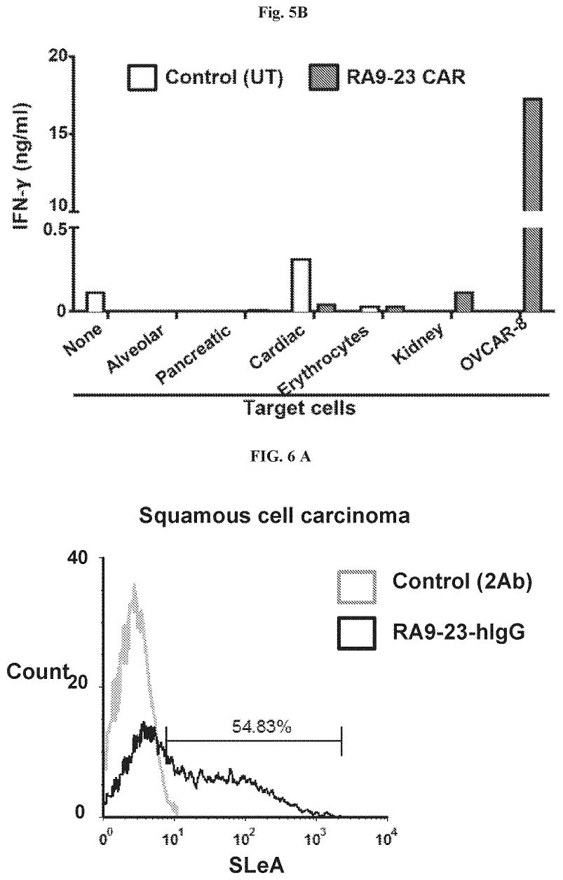

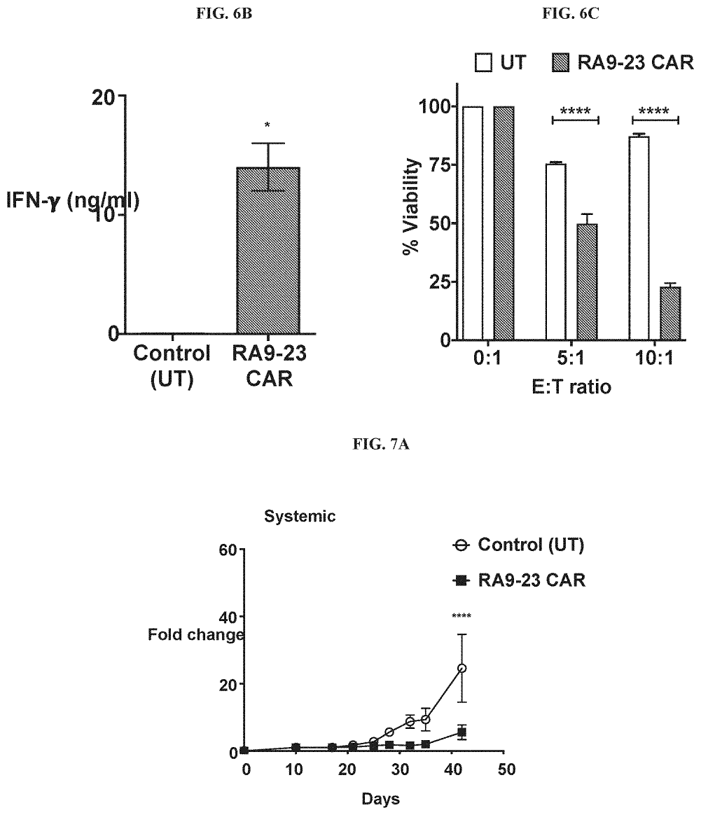

Next, the cytotoxicity potential of RA9-23 CAR T cells was evaluated on SLeA-expressing cancer cells. The FaDu pharynx squamous cell carcinoma cells were shown to express SLeA by staining with the RA9-23 monoclonal antibody (FIG. 6A). Then, the squamous cell carcinoma cells were co-cultured with RA9-23 CAR T cells what resulted in induction of IFN-γ cytokine secretion. There was no response when these cells were co-cultured with ustransduced T cells (FIG. 6B). Then, FaDu cells were then co-cultured with RA9-23 CAR T cells at different ratios and survival was monitored after 16 hours. A clear, dose-dependent specific killing of the SLeA-expressing target cells by RA9-23 CAR T cell was observed. Higher target (T) to effetor(E) ratio (T:E ratio) provided higher cytotoxicity; 50% and 80% killing were observed for 1:5 and 1:10 T:E ratio, respectively (FIG. 6C). Altogether, these data support the cytotoxic potential of RA9-23 CAR T cells against SLeA-expressing cells.

### Example 6. RA9-23 CAR T Cells Induce In Vivo Anti-Tumor Response

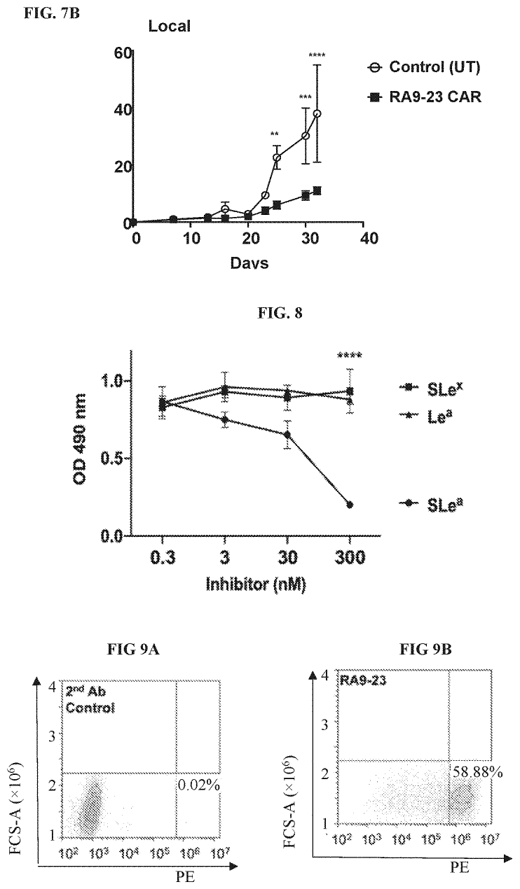

To assess in vivo cytotoxicity of RA9-23 CAR T cells, a xenograft model of pharynx squamous cell carcinoma was established in immune-compromised NOD-SCID-Gamma (NSG) mice by subcutaneous injection of FaDu cells into the flank. Once tumors were palpable, mice were irradiated, and then treated by a single dose systemic (intravenous) or local (intra-tumoral) adaptive transfer of RA9-23 CAR T cells or untransduced T cells (FIG. 7). Both types of RA9-23 CAR T administration (systemic or local) led to a significant reduction in tumor growth ˜25 days after tumor cell inoculation in comparison to the control mice that were treated with untransduced T cells, and tumor inhibition was maintained for ˜40 days (FIG. 7). Collectively, our study demonstrates the therapeutic potential of RA9-23 CAR T cells against SLeA-expressing cancer cells.

### Example 7. Expression of SLe^{a} in Different Types of Human Cancer

Immunohistochemistry of Human Cancers Tissue Microarray

The cloned RA9-23 human IgG1 antibody was biotinylated using the EZ-Link biotinylation Kit (Micro Sulfo-NHS-SS-Biotin; Pierce, Rockford, Ill.) according to the manufacturers instructions, then human cancers tissue microarray (TMA) slides (BioSB CA, USA) consisting of twenty-three 2 mm cores formalin-fixed paraffin-embedded tissues were stained with this Bio-RA9-23-hIgG antibody. For this purpose, the slides were first deparaffinated by incubation in xylene (Merck) for 15 min twice, then rehydrated by sequential 2 min washes with decreased percentage of ethanol in double distilled H₂O solution (100%, 95%, 90%, 80%, 70%, 50%, DDW), then washed twice in DDW. For antigen unmasking, slides were incubated for 15 min with 95° C. pre-heated HIER T-EDTA buffer pH 9 (Zymo), then transferred to DDW for additional 15 min, followed by rinsing in PBS pH 7.4 once. Slides were then blocked for one hour at room temperature (RT) by incubating with blocking solution (PBS pH 7.4, 0.1% Tween, 1% chicken ovalbumin [Sigma]). Biotin/avidin blocking was performed using a kit (Zotal), according to manufacturer's instructions. Slides were rinsed briefly with PBS, then fixed with 4% paraformaldehyde (PFA) for 10 min in RT, washed with PBST (PBS pH 7.4, 0.1% Tween) for 1 min, and incubated with 10 ng/μl Bio-RA9-23-hIgG overnight at 4° C. in a humidified chamber. The next day, slides were washed in PBST for 5 min, twice, then incubated with freshly prepared 0.3% H₂O₂ in PBS for 15 min. After one wash with PBS pH7.4, slides were incubated with 1 μg/ml HRP-streptavidin in PBS (Jackson) for 30 minutes at RT, followed by three washes with PBS 5 min each, then developed with substrate (3,3′-diaminobenzidine tetrahydrochloride; DAB) for 3 min, followed by washing once with DDW for 1 min and mounting with PermaMounter (Bio-SB). Slides were screened with Nikon eclipse Ti microscope at ×10 magnification.

Results

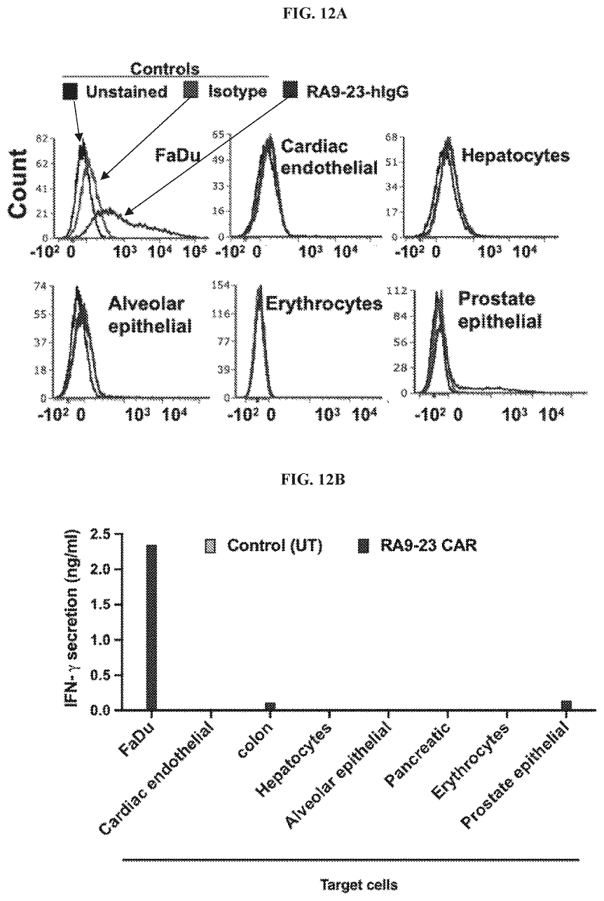

Human cancers tissue microarray (TMA) slides containing twenty three different cancer tissues were stained by immunohistochemistry using biotinylated RA9-23 antibody (Bio-RA9-23-hIgG) prepared as described above. The TMA included samples from melanoma, lung squamous cell carcinoma, lung adenocarcinoma, lung neuroendocrine cancer, papillary thyroid carcinoma, ductal breast carcinoma, Her-2 negative breast carcinoma, endometrial carcinoma, ovarian carcinoma, prostate adenocarcinoma, seminoma, hepatocellular carcinoma, renal clear cell carcinoma, diffuse type gastric adenocarcinoma, gastric GIST, pancreatic adenocarcinoma, colon adenocarcinoma, CLL/SLL lymphoma, follicular lymphoma, extranodal marginal zone lymphoma, mantle cell lymphoma, diffuse large B-cell lymphoma and lymphoblastic lymphoma. Of these tissues, lung and pancreatic adenocarcinomas showed strong staining, colon carcinoma and HER2-neg breast carcinoma showed moderate staining, and the other tissues seemed to be negative for SLeA. The results are presented in FIG. 11. As follows from these results, lung and pancreatic adenocarcinoma showed very high level of staining, and colon adenocarcinoma and Her-2 negative breast carcinoma showed high level of staining. This is a clear indication that these types of cancer express SLeA and may be targeted and treating using the CAR of the present invention that binds specifically to SLeA antigen.

### Example 8. RA9-23 CAR-T Safety

As discussed in Example 5, SLeA expression on human cells was evaluated by FACS using staining with RA9-23-hIgG. It revealed a strong SLeA expression in FaDu pharynx squamous cell carcinoma cell. Such expression was not revealed in other examined primary human cells (cardiac endothelial cells, colon, hepatocytes, alveolar epithelial cells, pancreatic, erythrocytes and kidney epithelial cells) as shown in FIG. 12A. Next, we evaluated off-target cytotoxicity of RA9-23 CAR T cells of the present invention by co-culturing RA9-23 CAR or untransduced T cells with the same human primary cells and measuring IFNγ secretion, using the SLeA-positive FaDu cells as a positive control. The results are presented in FIG. 12B. While FaDu cells showed high levels of IFNγ secretion, there was minimal IFNγ secretion in the co-culture media of either the RA9-23 CAR T cells or the untransduced T cells stimulated by primary normal cells. These results support very low risk for off-target cytotoxicity by the engineered CAR T cells.

### Example 9. A Study of Anti-Sialyl Lewis a CAR T Cells (RA9-23 CAR T Cells) in Patients with Solid Tumors

Summary

This clinical trial is an open-label, single-center, phase I study designed to investigate the safety and tolerability of a single infusion of autologous peripheral blood T-lymphocytes transduced with the anti-Sialyl Lewis A (SLeA; CA19.9) RA9-23 Chimeric Antigen Receptor Gene (RA9-23 CAR T cells). The primary aim of the trial is to evaluate the safety and tolerability of RA9-23 CAR T cells in patients with SLeA antigen-expressing, advanced solid tumors. The secondary aim of the trial is to assess the anti-tumor activity of RA9-23 CAR T cells in patients with SLeA antigen-expressing, advanced solid tumors. Patients aged 18 years or older with advanced solid tumors that consent to pre-screening that allows their tumors to be assessed for SLeA expression by immunohistochemistry. Patients whose tumors test positive for SLeA, are then allowed to proceed to eligibility screening and, if found to fulfil the eligibility criteria, are registered in the study. The study involves an initial dose escalation phase followed by an expansion phase.

Phase: Phase 1

### DETAILED DESCRIPTION

Autologous peripheral blood T-lymphocytes transduced with the RA9-23 CAR and administered to patients with SLeA expressing advanced solid tumors.

Aims: To evaluate the safety and tolerability of an intravenous infusion of autologous peripheral blood T-lymphocytes transduced with the RA9-23 CAR in patients with SLeA expressing advanced solid tumors.

Primary Objectives: To determine the maximum tolerated dose and rate of dose limiting toxicities of a single intravenous infusion of autologous peripheral blood T-lymphocytes transduced with the RA9-23 CAR in patients with SLeA expressing advanced solid tumors (RA9-23 CAR T cells).

Secondary Objectives: (1) To assess the anti-tumor activity of the RA9-23 CAR T cells in terms of overall response, duration of response, progression free survival and overall survival. (2) To assess persistence in peripheral blood of the RA9-23 CAR T cells.

Patients (12) are recruited for the study of the dose escalation phase consisting of 4 dose levels, each with dose level cohorts of 3 patients. Following completion of the dose-escalation phase, additional patients with SLeA-expressing solid tumors are recruited to the study. These patients are administered the maximum number of cells safely delivered in the dose escalation phase of the study. A subset comprising 5 patients in the expansion cohort are administered Indium-111 labelled T-cells and imaged by SPECT to determine the biodistribution of reinfused T cells.

If the proposed number of T cells is unable to be obtained due to technical production reasons, the available number are infused.

Up to 30 patients are be treated on this protocol.

Study Type: Interventional (Clinical Trial)

Intervention Model: Sequential Assignment

Intervention Model Description: The study employs dose level cohorts of three patients that treated at each level, based on the number of T cells to be infused using the “3+3” dose-escalation strategy. If the proposed number of T cells is unable to be obtained due to technical production reasons, the available number is infused. However, the cohort is only escalated when a minimum of three patients have been safely treated at the planned level.

The fourth cohort dose level is set according to the maximum level considered to be feasible taking into account technical constraints. The study is expanded to accrue additional patients at the maximul tolerated dose (expansion phase cohort) with an expansion cohort of upto 20 patients. For technical and logistic reasons the maximum number of patients to be enrolled on the study will be 30.

Masking: None (Open Label)

Primary Purpose: Treatment

Although the present invention has been described hereinabove by way of preferred embodiments thereof, it can be modified, without departing from the spirit and nature of the subject invention as defined in the appended claims

1-53. (canceled)

54. A chimeric antigen receptor (CAR) comprising an antigen binding domain that binds specifically to Sialyl Lewis A glycan (SLeA), wherein the antigen binding domain comprises three complementarity determining regions (CDRs) and four framework (FR) domains of a heavy-chain variable domain (VH) having amino acid sequence as set forth in SEQ ID NO: 1 and three CDRs and four FRs of a light-chain variable domain (VL) having amino acid sequence as set forth in SEQ ID NO: 4, or an analog thereof having at least 90% sequence identity to said sequences.

55. The CAR according to claim 53, wherein the VH-CDR1 comprises amino acid sequence selected from SEQ ID NOs: 6 and 30, the VH-CDR2 comprises amino acid sequence selected from 7 and 31, the VH-CDR3 comprises amino acid sequence SEQ ID NO: 8, and CDRs 1, 2, and 3 of the VL domain comprise amino acid sequences SEQ ID NOs: 9, 10, and 11, respectively.

56. The CAR according to claim 55, wherein the CAR is characterized by at least one of:
(i) the VH-CDR2 comprises at least one non-conservative substitution;
(ii) the VH-CDR2 comprises amino acid sequence selected from SEQ ID NO: 12 and 36;
(iii) the VH domain comprises amino acid sequence set forth in SEQ ID NO: 2 and the VL domain comprises amino acid sequence SEQ ID NO: 4;
(iv) wherein the antigen binding domain further comprises at least 2 or 3, or 4 non-conservative substitutions of amino acids in the framework sequence of a domain selected from (a) the VH domain; (b) the VL domain; and (c) both VH and VL domains.

57. The CAR according to claim 56, characterized by at least one of: (i) at least 2 of said non-conservative substitutions is for proline amino acid residue; and (ii) the non-conservative substitutions are at a position selected from positions 1, 110, 114 of SEQ ID NO: 1 or 2, position 22 of SEQ ID NO: 4 and any combination thereof.

58. The CAR according to claim 57, wherein the (i) antigen binding domain comprises a substitution of the amino acid at position 1 of SEQ ID NO: 1 or of SEQ ID NO: 2 for a positively charged amino acid residue or (ii) antigen binding domain comprises a substitution of the amino acid at position 1 of SEQ ID NO: 1 or of SEQ ID NO: 2 for a positively charged amino acid residue selected from Lys and Arg.

59. The CAR according to claim 53, wherein:
(i) the VH-CDR 1, 2 and 3 comprise amino acid sequences SEQ ID NOs: 30, 12, and 8, respectively, the VL-CDRs 1, 2 and 3 comprise amino acid sequences SEQ ID NOs: 9, 10 and 11, respectively, VH-FRs 1, 2 and 4 comprise amino acid sequences SEQ ID NOs: 39, 42 and 43, respectively, and the VL-FR 1 comprises acid sequences SEQ ID NO: 44;
(ii) the CDRs 1, 2, and 3 of the VH domain comprises amino acid sequences SEQ ID NOs: 30, 36 and 8, respectively, the CDRs 1, 2, and 3 of the VL domain comprise amino acid sequences SEQ ID NOs: 9, 10 and 11, the VH-FRs 1, 2 and 4 comprise amino acid sequences SEQ ID NOs: 40, 42 and 43, respectively, and the VL-FR1 comprises amino acid sequences SEQ ID NO: 44;
(iii) the CDRs 1, 2, and 3 of the VH domain comprise amino acid sequences SEQ ID NOs: 30, 36 and 8, respectively, the CDRs 1, 2, and 3 of the VL domain comprise amino acid sequences SEQ ID NOs: 9, 10 and 11, respectively, the VH-FRs 1, 2, 3 and 4 comprise amino acid sequences SEQ ID NOs: 40, 42, 45 and 43, respectively, and the VL-FR1, 2, 3 and 4 comprise amino acid sequences SEQ ID NOs: 44, 46, 47 and 48, respectively; or
(iv) the VH domain comprises amino acid sequence SEQ ID NO: 3 and the VL domain comprises amino acid sequence SEQ ID NO: 5.

60. The CAR according to claim 53, characterized by at least one of:
(i) the CAR further comprising at least one conservative substitution in the framework(s) of the VH domain and/or VL domain, wherein the resulted VH domain has at least 90% sequence identity to SEQ ID NO: 3 and/or the resulted VL domain has at least 90% sequence identity to SEQ ID NO: 5;
(ii) the VH and the VL domains are linked by a spacer to form a single chain variable fragment (scFv); and
(iii) wherein the VH and the VL domains are linked by a spacer to form a scFv, wherein the spacer comprises amino acid sequence comprising from 2 to 6 repetitions of the amino acid sequence set forth in SEQ ID NO: 16.

61. The CAR according to claim 60, wherein the scFv comprises amino acid sequence selected from SEQ ID NO: 15 and 37 or an analog thereof having at least 90% sequence identity to said sequences.

62. The CAR according to claim 53, wherein the CAR comprises a transmembrane domain (TM domain), a costimulatory domain and an activation domain.

63. The CAR according to claim 62, wherein the CAR is characterized by at least one of:
(i) the TM domain is a TM domain of a receptor selected from CD28 and CD8, or an analog thereof having at least 85% amino acid identity to said sequences;
(ii) the costimulatory domain is selected from a costimulatory domain of a protein selected from CD28, 4-1BB, OX40, iCOS, CD27, CD80, and CD70, an analog thereof having at least 85% amino acid identity to the original sequence and any combination thereof;
(iii) the TM domain and the costimulatory domain are both derived from CD28;
(iv) wherein the antigen binding domain is linked to the TM domain via a spacer;
(v) wherein the activation domain is selected from FcRγ and CD3-ζ activation domains; and
(vi) further comprising a leading peptide.

64. The CAR according to claim 63, wherein the CAR is characterized by at least one of:
(i) the TM domain and the costimulatory domain have amino acid sequence SEQ ID NO: 17 or an analog thereof having at least 85% amino acid identity to the sequence;
(ii) the spacer comprises amino acid sequence comprising from 1 to 4 repetitions of amino acid sequence SEQ ID NO: 16;
(iii) the activation domain is FcRγ having amino acid sequence SEQ ID NO: 18 or an analog thereof having at least 85% amino acid identity to the original sequence; and
(vi) the leading peptide has amino acid sequence SEQ ID NO: 19 or an analog thereof having at least 85% amino acid identity.

65. The CAR according to claim 53, comprising amino acid sequence selected from SEQ ID NO: 20 and 38.

66. A nucleic acid molecule, encoding the CAR according to claim 53 or the construct or vector comprising the nucleic acid molecule.

67. The nucleic acid molecule according to claim 66, wherein the nucleic acid molecule is characterized by at least one of:
(i) the nucleic acid molecule encodes amino acid sequence selected from SEQ ID NO: 3, SEQ ID NO: 5 and both SEQ ID NOs: 3 and 5;
(ii) the nucleic acid molecule comprises a nucleic acid sequence selected from SEQ ID NO: 13, SEQ ID NO: 14, a variant of SEQ ID NO: 13 or 14 having at least 95% sequence identity to the sequence(s), and a combination thereof
(iii) the nucleic acid molecule encodes amino acid sequence SEQ ID NO: 15;
(iv) the nucleic acid molecule comprises nucleic acid sequence SEQ ID NO: 21 or a variant thereof having at least 95% sequence identity to said sequence;
(v) the nucleic acid molecule encodes amino acid sequence selected from the group consisting of SEQ ID NOs: 17, 18, 19, an analog thereof, and any combination thereof;
(vi) the nucleic acid molecule comprises a nucleic acid sequence selected from the group consisting of SEQ ID NO: 22, 23, 24, a variant thereof having at least 95% sequence identity to said sequence(s), and a combination thereof;
(vi) the nucleic acid molecule encodes amino acid sequence selected from the group consisting of SEQ ID NO: 20, SEQ ID NO: 28, and SEQ ID NO: 38;
(vii) the nucleic acid molecule comprises a nucleic acid sequence selected from the group consisting of SEQ ID NO: 25, SEQ ID NO: 29, and a variant thereof having at least 95% sequence identity to the original sequence.

68. A cell comprising the CAR according to claim 54 or the nucleic acid molecule encoding same or the construct or vector comprising the nucleic acid molecule.

69. The cell according to claim 56, wherein the cell is characterized by at least one of:
(i) the cell expresses or capable of expressing the CAR; and
(ii) the cell is selected from a T cell and a natural killer (NK) cell.

70. T cells comprising the CAR according to claim 54 or the nucleic acid encoding the CAR.

71. A pharmaceutical composition comprising a plurality of cells according claim 68, and a pharmaceutically acceptable carrier.

72. A method for treating cancer in a subject in need thereof comprising administering a therapeutically effective amount of cells according to claim 68 or a pharmaceutical composition comprising same.

73. A method according to claim 72, wherein the cancer is selected from lung adenocarcinoma, pancreatic adenocarcinoma, colon adenocarcinoma, Her-2 negative breast carcinoma and pharynx squamous cell carcinoma.
